(19) United States
(12) Patent Application Publication
(10) Pub. No.: US 2026/0229335 A1
(43) Pub. Date: Aug. 6, 2026

(54) SYSTEM, METHOD, AND COMPUTER PROGRAM PRODUCT FOR OPERATING A FLUID INJECTION SYSTEM

(71) Applicant: Bayer HealthCare LLC, Whippany, NJ (US)

(72) Inventor: Sri SHRIRAM, Murrysville, PA (US)

(21) Appl. No.: 19/159,896

(22) PCT Filed: Mar. 13, 2023
(86) PCT No.: PCT/US2023/015065

§ 371 Date: Aug. 26, 2025

(51) Int. Cl. G16H 20/17; G16H 10/60

(52) U.S. Cl. CPC G16H 20/17; G16H 10/60

(57) ABSTRACT

Systems for operating a fluid injection system may include at least one processor to receive patient data associated with a patient undergoing a fluid injection procedure, automatically generate at least one parameter of an injection protocol for the fluid injection procedure based on the patient data and independent of an input associated with a dosage of a medical fluid for the fluid injection procedure received via a user interface associated with a fluid injection system that is to execute the fluid injection procedure, wherein, when automatically generating the at least one parameter, the at least one processor is to determine the dosage of the medical fluid for the fluid injection procedure, and provide the at least one parameter of the injection protocol to the fluid injection system. Methods and computer program products are also disclosed.

### BACKGROUND

### 1. Field

This disclosure relates generally to control systems and/or devices associated with medical devices and, in some non-limiting embodiments, to systems, methods, and computer program products for operating fluid injection systems.

### 2. Technical Considerations

An injection device, such as a fluid injection device (e.g., a medical fluid delivery device) may be used by a medical practitioner, such as a physician, in a medical diagnostic procedure and/or a medical therapeutic procedure. For example, the medical practitioner may use the fluid injection device to inject a patient with one or more medical fluids. The fluid injection device may be used for pressurized injection of a medical fluid, such as a radiological contrast material (e.g., a contrast agent, a radiocontrast agent, contrast media, etc.), and/or a flushing agent, such as saline, in medical imaging procedures, such as angiography, computed tomography (CT), ultrasound, magnetic resonance imaging (MRI), and positron emission tomography (PET). In some instances, the fluid injection device is designed to deliver an amount of a medical fluid at one or more flow rates into a patient.

### SUMMARY

Accordingly, provided are systems, methods, and computer program products for operating a fluid injection system that improve the efficiency and accuracy of generating an injection protocol for delivery of a medical fluid to a patient during a fluid injection procedure.

Further non-limiting embodiments or aspects are set forth in the following numbered clauses:

Clause 1: A system for operating a fluid injection system comprising: at least one processor programmed or configured to: receive patient data associated with a patient undergoing a fluid injection procedure, wherein the patient data comprises data associated with a weight of the patient; automatically generate at least one parameter of an injection protocol for the fluid injection procedure based on the patient data and independent of an input associated with a dosage of a medical fluid for the fluid injection procedure received via a user interface associated with a fluid injection system that is to execute the fluid injection procedure, wherein, when automatically generating the at least one parameter, the at least one processor is programmed or configured to: determine the dosage of the medical fluid for the fluid injection procedure based on: the weight of the patient, a concentration of the medical fluid, an anatomical region of the patient in which the fluid injection procedure is to take place, or any combination thereof; and provide the at least one parameter of the injection protocol to the fluid injection system.

Clause 2: The system of clause 1, further comprising a display unit, wherein the at least one processor is further programmed or configured to: cause a plurality of patient identifiers to be displayed in a user interface on the display unit; and receive a selection of a patient identifier of the plurality of patient identifiers via the user interface; wherein, when receiving the patient data, the at least one processor is programmed or configured to: receive the patient data based on the selection of the patient identifier received via the user interface.

Clause 3: The system of clauses 1 or 2, wherein, when receiving the patient data, the at least one processor is programmed or configured to: receive the patient data from a user input via a user interface displayed on a display unit.

Clause 4: The system of any of clauses 1-3, wherein the patient data associated with the patient undergoing the fluid injection procedure corresponds to a patient record for the patient stored in a hospital information system, and wherein the at least one processor is further programmed or configured to: transmit a unique identifier associated with the patient record for the patient to the hospital information system; and wherein, when receiving the patient data, the at least one processor is programmed or configured to: receive the patient data from the hospital information system.

Clause 5: The system of any of clauses 1-4, wherein, when automatically generating the at least one parameter of the injection protocol for the fluid injection procedure, the at least one processor is further programmed or configured to: determine a first dosage of the medical fluid for a first injection phase of the fluid injection procedure; determine a first flush phase of the fluid injection procedure, wherein the first flush phase comprises a first injection of saline fluid; determine a second dosage of the medical fluid for a second injection phase of the fluid injection procedure based on at least one of the following: the weight of the patient, the concentration of the medical fluid, the anatomical region, or any combination thereof; and determine a second flush phase of the fluid injection procedure, wherein the second flush phase comprises a second injection of saline fluid.

Clause 6: The system of any of clauses 1-5, wherein the at least one processor is further programmed or configured to: cause the fluid injection procedure to be executed in order of the first injection phase, the first flush phase, a hold phase, the second injection phase, and the second flush phase.

Clause 7: The system of any of clauses 1-6, wherein, when automatically generating the at least one parameter of the injection protocol for the fluid injection procedure, the at least one processor is programmed or configured to: determine a first flow rate of the medical fluid for the first injection phase based on the concentration of the medical fluid and the anatomical region; and determine a second flow rate of the medical fluid for the second injection phase based on the concentration of the medical fluid and the anatomical region.

Clause 8: The system of any of clauses 1-7, further comprising a display unit, wherein the at least one processor is further programmed or configured to: cause a plurality of anatomical region identifiers to be displayed in a user interface on the display unit; and receive a selection of an anatomical region identifier of the plurality of anatomical region identifiers via the user interface; wherein, when automatically generating the at least one parameter of the injection protocol for the fluid injection procedure, the at least one processor is programmed or configured to: determine a type of the medical fluid, a concentration of the medical fluid, and the anatomical region of the patient in which the fluid injection procedure is to take place based on the selection of the anatomical region identifier received via the user interface.

Clause 9: A method for operating a fluid injection system, comprising: receiving, with at least one processor, patient data associated with a patient undergoing a fluid injection procedure, wherein the patient data comprises data associated with a weight of the patient; automatically generating, with at least one processor, at least one parameter of an injection protocol for the fluid injection procedure and independent of an input associated with a dosage of a medical fluid for the fluid injection procedure received via a user interface associated with a fluid injection system that is to execute the fluid injection procedure based on the patient data, wherein automatically generating the at least one parameter comprises: determining the dosage of the medical fluid for the fluid injection procedure based on: the weight of the patient, a concentration of the medical fluid, or an anatomical region of the patient in which the fluid injection procedure is to take place; or any combination thereof; and providing, with at least one processor, the at least one parameter of the injection protocol.

Clause 10: The method of clause 9, further comprising: causing a plurality of patient identifiers to be displayed in a user interface on a display unit; and receiving a selection of a patient identifier of the plurality of patient identifiers via the user interface; wherein receiving the patient data comprises: receiving the patient data based on the selection of the patient identifier received via the user interface.

Clause 11: The method of clauses 9 or 10, wherein the patient data associated with the patient undergoing the fluid injection procedure corresponds to a patient record for the patient stored in a hospital information system, and wherein the method further comprises: transmitting a unique identifier associated with the patient record for the patient to the hospital information system; and wherein receiving the patient data comprises: receiving the patient data from the hospital information system.

Clause 12: The method of any of clauses 9-11, wherein receiving the patient data comprises: receiving the patient data from a user input via a user interface displayed on a display unit.

Clause 13: The method of any of clauses 9-12, wherein automatically generating the at least one parameter of the injection protocol for the fluid injection procedure further comprises: determining a first dosage of the medical fluid for a first injection phase of the fluid injection procedure; determining a first flush phase of the fluid injection procedure comprising a first injection of a saline fluid; determining a second dosage of the medical fluid for a second injection phase of the fluid injection procedure based on at least one of the following: the weight of the patient, the concentration of the medical fluid, or the anatomical region; and determining a second flush phase comprising a second injection of the saline fluid.

Clause 14: The method of any of clauses 9-13, further comprising: causing execution of the fluid injection procedure in order of the first injection phase, the first flush phase, a hold phase, the second injection phase, and the second flush phase.

Clause 15: The method of any of clauses 9-14, wherein automatically generating the at least one parameter of the injection protocol for the fluid injection procedure further comprises: determining a first flow rate of the medical fluid for the first injection phase based on the concentration of the medical fluid and the anatomical region of the patient in which the fluid injection procedure is to take place; and determining a second flow rate of the medical fluid for the second injection phase based on the concentration of the medical fluid and the anatomical region of the patient in which the fluid injection procedure is to take place.

Clause 16: The method of any of clauses 9-15, further comprising: causing a plurality of anatomical region identifiers to be displayed in a user interface on a display unit; and receiving a selection of an anatomical region identifier of the plurality of anatomical region identifiers via the user interface; wherein automatically generating the at least one parameter of the injection protocol for the fluid injection procedure comprises: determining the medical fluid, the concentration of the medical fluid, and the anatomical region of the patient in which the fluid injection procedure is to take place based on the selection of the anatomical region identifier received via the user interface.

Clause 17: A computer program product for operating a fluid injection system, the computer program product comprising at least one non-transitory computer-readable medium including one or more instructions that, when executed by at least one processor, cause the at least one processor to: receive patient data associated with a patient undergoing a fluid injection procedure, wherein the patient data comprises data associated with a weight of the patient; automatically generate at least one parameter of an injection protocol for the fluid injection procedure based on the patient data and independent of an input associated with a dosage of a medical fluid for the fluid injection procedure received via a user interface associated with a fluid injection system that is to execute the fluid injection procedure, wherein, the one or more instructions that cause the at least one processor to automatically generate the at least one parameter, cause the at least one processor to: determine the dosage of the medical fluid for the fluid injection procedure based on at least one of the following: the weight of the patient, a concentration of the medical fluid, or an anatomical region of the patient in which the fluid injection procedure is to take place; and provide the at least one parameter of the injection protocol to the fluid injection system.

Clause 18: The computer program product of clause 17, wherein the one or more instructions further cause the at least one processor to: cause a plurality of patient identifiers to be displayed in a user interface on a display unit; and receive a selection of a patient identifier of the plurality of patient identifiers via the user interface; wherein, the one or more instructions that cause the at least one processor to receive the patient data, cause the at least one processor to: receive the patient data based on the selection of the patient identifier received via the user interface.

Clause 19: The computer program product of clauses 17 or 18, wherein the patient data associated with the patient undergoing the fluid injection procedure corresponds to a patient record for the patient stored in a hospital information system, and wherein the one or more instructions further cause the at least one processor to: transmit a unique identifier associated with the patient record for the patient to the hospital information system; and wherein, the one or more instructions that cause the at least one processor to receive the patient data, cause the at least one processor to: receive the patient data from the hospital information system.

Clause 20: The computer program product of any of clauses 17-19, wherein, the one or more instructions that cause the at least one processor to automatically generate the at least one parameter of the injection protocol for the fluid injection procedure further cause the at least one processor to: determine a first dosage of the medical fluid for a first injection phase of the fluid injection procedure; determine a first flush phase of the fluid injection procedure, wherein the first flush phase comprises a first injection of saline fluid; determine a second dosage of the medical fluid for a second injection phase of the fluid injection procedure based on at least one of the following: the weight of the patient, the concentration of the medical fluid, the anatomical region, or any combination thereof; and determine a second flush phase of the fluid injection procedure, wherein the second flush phase comprises a second injection of saline fluid; and wherein the one or more instructions further cause the at least one processor to: cause the fluid injection procedure to be executed in order of the first injection phase, the first flush phase, a hold phase, the second injection phase, and the second flush phase.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the present disclosure. As used in the specification and the claims, the singular form of “a,” “an,” and “the” include plural referents unless the context clearly dictates otherwise.

### BRIEF DESCRIPTION OF THE DRAWINGS

Additional advantages and details of non-limiting embodiments or aspects are explained in greater detail below with reference to the exemplary embodiments that are illustrated in the accompanying schematic figures, in which:

FIG. 1A is a diagram of a non-limiting embodiment of an environment in which systems, devices, products, apparatus, and/or methods, described herein, may be implemented, according to the principles of the present disclosure;

FIG. 1B is a diagram of a non-limiting embodiment of a system for automatically generating an injection protocol for delivery of a medical fluid to a patient during a fluid injection procedure;

FIG. 2 is a diagram of a non-limiting embodiment of components of one or more systems or one or more devices of FIGS. 1A and 1B;

FIG. 3 is a flowchart of a non-limiting embodiment of a process for operating a fluid injection system;

FIG. 4 is an illustration of a non-limiting embodiment of a user interface on a display screen, for use in a method for operating a fluid injection system;

FIG. 5 is an illustration of a non-limiting embodiment of a user interface on a display screen, for use in a method for operating a fluid injection system;

FIG. 6A is an illustration of a non-limiting embodiment of a user interface on a display screen, for use in a method for operating a fluid injection system;

FIG. 6B is an illustration of a non-limiting embodiment of a user interface on a display screen, for use in a method for operating a fluid injection system;

FIG. 7A is an illustration of a non-limiting embodiment of a user interface on a display screen, for use in a method for operating a fluid injection system;

FIG. 7B is an illustration of a non-limiting embodiment of a user interface on a display screen, for use in a method for operating a fluid injection system;

FIG. 7C is an illustration of a non-limiting embodiment of a user interface on a display screen, for use in a method for operating a fluid injection system;

FIG. 7D is an illustration of a non-limiting embodiment of a user interface on a display screen, for use in a method for operating a fluid injection system;

FIG. 7E is an illustration of a non-limiting embodiment of a user interface on a display screen, for use in a method for operating a fluid injection system;

FIG. 8 is a diagram of a non-limiting embodiment of an environment including a workstation device and a fluid injection system;

FIG. 9 is a diagram of a non-limiting embodiment of a fluid injection system; and

FIG. 10 is a diagram of a non-limiting embodiment of an injector head unit of a fluid injection system.

### DETAILED DESCRIPTION

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the present disclosure as it is oriented in the drawing figures. However, it is to be understood that the present disclosure may assume various alternative variations and step sequences, except where expressly specified to the contrary. It is also to be understood that the specific devices and processes illustrated in the attached drawings, and described in the following specification, are simply exemplary embodiments of the present disclosure. Hence, specific dimensions and other physical characteristics related to the embodiments or aspects of the embodiments disclosed herein are not to be considered as limiting unless otherwise indicated.

No aspect, component, element, structure, act, step, function, instruction, and/or the like used herein should be construed as critical or essential unless explicitly described as such. Also, as used herein, the articles “a” and “an” are intended to include one or more items, and may be used interchangeably with “one or more” and “at least one.” Furthermore, as used herein, the term “set” is intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise.

As used herein, the terms “communication” and “communicate” may refer to the reception, receipt, transmission, transfer, provision, and/or the like of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit (e.g., a third unit located between the first unit and the second unit) processes information received from the first unit and communicates the processed information to the second unit. In some non-limiting embodiments, a message may refer to a network packet (e.g., a data packet and/or the like) that includes data. It will be appreciated that numerous other arrangements are possible.

As used herein, the term “system” may refer to one or more computing devices or combinations of computing devices such as, but not limited to, processors, servers, client devices, software applications, and/or other like components. In addition, reference to “a server” or “a processor,” as used herein, may refer to a previously-recited server and/or processor that is recited as performing a previous step or function, a different server and/or processor, and/or a combination of servers and/or processors. For example, as used in the specification and the claims, a first server and/or a first processor that is recited as performing a first step or function may refer to the same or different server and/or a processor recited as performing a second step or function.

In some instances, a fluid injection system may be a part of a group of devices that are used in a medical treatment facility, such as a hospital, during treatment of patients, and/or in a medical imaging facility. Individuals, such as medical care providers (e.g., personnel that operate magnetic resonance imaging (MRI) devices), may use a workstation device to provide parameters for an injection protocol for a fluid injection procedure to be administered by the fluid injection system. The parameters may be provided as inputs that are manually provided by a user. However, an injection protocol may need to be customized to a specific patient that is undergoing the fluid injection procedure, so that associated medical care and/or evaluations are performed correctly, and/or so that the patient is not harmed by the injection. For example, certain medical fluids to be injected in a fluid injection procedure may need to be correctly dosed based on the patient and injected at a specific flow rate for best results.

The described systems, methods, and computer program products improve the accuracy and efficiency of generating injection protocols and operating fluid injection procedures of a fluid injection system. In some non-limiting embodiments, an injection protocol management system may receive patient data associated with a patient undergoing a fluid injection procedure, automatically generate at least one parameter of an injection protocol for the fluid injection procedure based on the patient data, and provide the at least one parameter of the injection protocol to a fluid injection system. In some non-limiting embodiments, the patient data may include data associated with a weight of the patient. In some non-limiting embodiments, when automatically generating the at least one parameter, the injection protocol management system may determine a first dosage of a medical fluid for the fluid injection procedure based on at least one of: the weight of the patient, a concentration of the medical fluid, or an anatomical region of the patient in which the fluid injection procedure is to take place.

In some non-limiting embodiments, the injection protocol management system may include a display unit and the injection protocol management system may cause a plurality of patient identifiers to be displayed in a user interface on the display unit and then receive a selection of a patient identifier of the plurality of patient identifiers via the user interface. In some non-limiting embodiments, the patient identifier may include a unique identifier associated with a patient record, and receiving the patient data based on the selection of the patient identifier received via the user interface may include retrieving the patient data from the patient record stored in a hospital information system. In some non-limiting embodiments, the patient identifier may include an identifier that is unassociated with a patient record, and the injection protocol management system may receive patient data based on the selection of the patient identifier received via the user interface. In some non-limiting embodiments, the injection protocol management system may receive the patient data from a user input via the user interface.

In some non-limiting embodiments, when determining the first dosage of the medical fluid for the fluid injection procedure, the injection protocol management system may determine the first dosage of the medical fluid for a first injection phase of the fluid injection procedure, and when automatically generating the at least one parameter of the injection protocol for the fluid injection procedure, the injection protocol management system may determine a first flush phase of the fluid injection procedure including a first injection of a saline fluid, determine a second dosage of the medical fluid for a second injection phase of the fluid injection procedure based on at least one of the following: the weight of the patient, the concentration of the medical fluid, or the anatomical region, and the injection protocol management system may determine a second flush phase including a second injection of the saline fluid. In some non-limiting embodiments, the injection protocol management system may execute the fluid injection procedure in order of the first injection phase, the first flush phase, a hold phase, the second injection phase, and the second flush phase. In some non-limiting embodiments, when automatically generating the at least one parameter of the injection protocol for the fluid injection procedure, the injection protocol management system may determine a first flow rate of the medical fluid for the first injection phase and a second flow rate of the medical fluid for the second injection phase based on the concentration of the medical fluid and the anatomical region.

In some non-limiting embodiments, the injection protocol management system may further include a display unit, and the injection protocol management system may cause a plurality of anatomical region identifiers to be displayed in the user interface on the display unit. In some non-limiting embodiments, the injection protocol management system may receive a selection of an anatomical region identifier of the plurality of anatomical region identifiers via the user interface. In some non-limiting embodiments, when automatically generating the at least one parameter of the injection protocol for the fluid injection procedure, the injection protocol management system may determine a type (e.g., a name, an identifier, etc.) of the medical fluid, the concentration of the medical fluid, and the anatomical region based on the selection of the anatomical region identifier received via the user interface.

In this way, non-limiting embodiments of the present disclosure provide for faster generation of injection protocols in fewer user steps, while also assuring accurate parameters of injection protocols through the automatic generation of injection protocol parameters based on patient data. Additional computational improvements are provided by integrating the retrieval of patient data from hospital information systems, from which the injection protocol parameters can be automatically generated (e.g., thereby reducing network resources, such as calls to devices that may otherwise be necessary for retrieving patient data in a process flow for generating an injection protocol, rather than requiring a user to retrieve patient data with a separate computing device, application, and/or process flow). Furthermore, patient safety and medical procedure efficacy are improved by automatically generating injection protocol parameters based on patient data and/or selected anatomical regions, by which the following parameters may be automatically determined: the medical fluid, the concentration of the medical fluid, the flow rate, and/or the like. This reduces the number of opportunities for human error to be introduced during setup of an injection protocol for a fluid injection procedure.

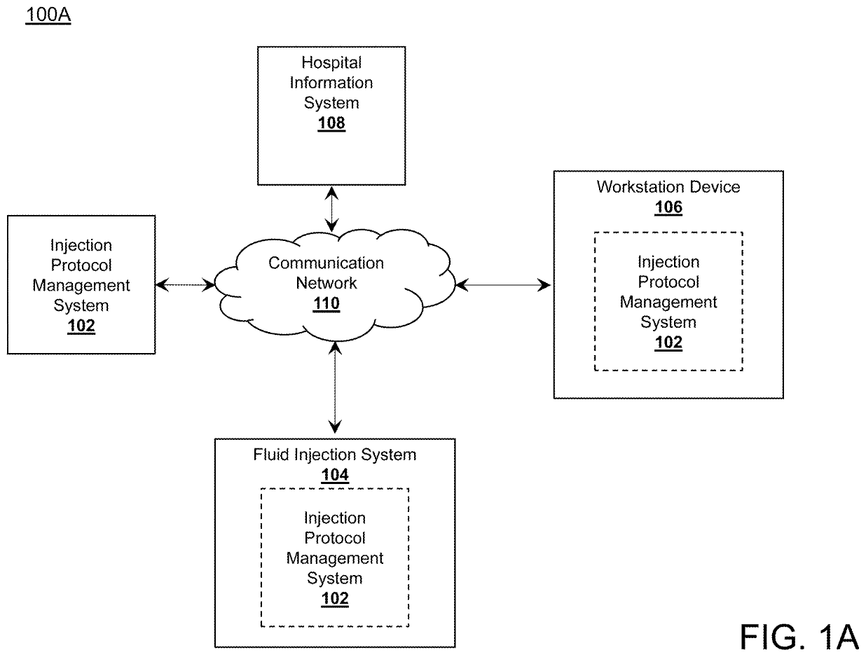

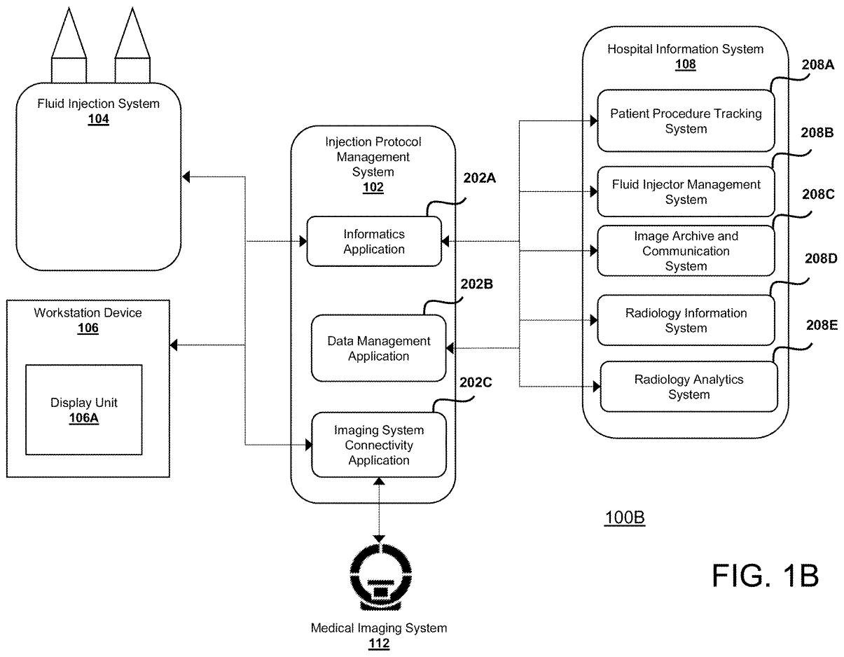

Referring now to FIG. 1A, FIG. 1A is a diagram of a non-limiting embodiment of an environment 100A in which devices, systems, methods, and/or computer program products, described herein, may be implemented. As shown in FIG. 1A, environment 100A includes injection protocol management system 102, fluid injection system 104, workstation device 106, hospital information system 108, and communication network 110. In some non-limiting embodiments, injection protocol management system 102, fluid injection system 104, workstation device 106, and/or hospital information system 108, may interconnect (e.g., establish a connection to communicate) via wired connections, wireless connections, or a combination of wired and wireless connections. Any devices or systems in environment 100A may communicate with each other in a same or different communication network 110 as other devices or systems.

In some non-limiting embodiments, injection protocol management system 102 may include one or more devices capable of being in communication with fluid injection system 104, workstation device 106, and/or hospital information system 108, via communication network 110. For example, injection protocol management system 102 may include one or more computing devices, such as one or more computers, one or more servers (e.g., a cloud server, a group of servers, etc.), one or more desktop computers, one or more mobile devices (e.g., one or more tablets, one or more smartphones, etc.), and/or the like. In some non-limiting embodiments, injection protocol management system 102 may include one or more (e.g., a plurality of) applications (e.g., software applications) that perform a set of functionalities on an external application programming interface (API) that allows injection protocol management system 102 to send data to an external system associated with the external API and to receive data from the external system associated with the external API. In some non-limiting embodiments, the application may be supported by an application associated with fluid injection system 104 that would allow injection protocol management system 102, which may function as a control room display, to be the only one device that controls fluid injection system 104 and, in such an example, injection protocol management system 102 may provide an authentication function. In some non-limiting embodiments, injection protocol management system 102 may be a component of fluid injection system 104 and/or workstation device 106.

In some non-limiting embodiments, fluid injection system 104 may include one or more devices capable of being in communication with injection protocol management system 102, workstation device 106, and/or hospital information system 108, via communication network 110. For example, fluid injection system 104 may include one or more computing devices, such as one or more computers, one or more servers (e.g., a cloud server, a group of servers, etc.), one or more desktop computers, one or mobile devices (e.g., one or more tablets, one or more smartphones, etc.), and/or the like. In some non-limiting embodiments, fluid injection system 104 may include one or more injection devices (e.g., one or more fluid injection devices, one or more fluid injectors). In some non-limiting embodiments, fluid injection system 104 is configured to administer (e.g., inject, deliver, etc.) contrast fluid including a contrast agent to a patient, and/or administer an aqueous fluid, such as saline, to a patient before, during, and/or after administering the contrast fluid. For example, fluid injection system 104 can inject one or more prescribed dosages of contrast fluid directly into a patient's blood stream via a hypodermic needle and syringe. In some non-limiting embodiments, fluid injection system 104 may be configured to continually administer the aqueous fluid to a patient through a peripheral intravenous line (PIV) and catheter, and one or more prescribed dosages of contrast fluid may be introduced into the PIV and administered via the catheter to the patient. In some non-limiting embodiments, fluid injection system 104 is configured to inject a dose of contrast fluid followed by administration of a particular volume of the aqueous fluid. In some non-limiting embodiments, fluid injection system 104 may include one or more exemplary fluid injection devices that are disclosed in: U.S. patent application Ser. No. 09/715,330, filed on Nov. 17, 2000, issued as U.S. Pat. No. 6,643,537; U.S. patent application Ser. No. 09/982,518, filed on Oct. 18, 2001, issued as U.S. Pat. No. 7,094,216; U.S. patent application Ser. No. 10/825,866, filed on Apr. 16, 2004, issued as U.S. Pat. No. 7,556,619; U.S. patent application Ser. No. 12/437,011, filed May 7, 2009, issued as U.S. Pat. No. 8,337,456; U.S. patent application Ser. No. 12/476,513, filed Jun. 2, 2009, issued as U.S. Pat. No. 8,147,464; and U.S. patent application Ser. No. 11/004,670, filed on Dec. 3, 2004, issued as U.S. 8,540,698, the disclosures of each of which are incorporated herein by reference in their entireties. In some non-limiting embodiments, fluid injection system 104 may include the MEDRAD® Stellant CT Injection System, the MEDRAD® Stellant FLEX CT Injection System, the MEDRAD® MRXperion MR Injection System, the MEDRAD® Mark 7 Arterion Injection System, the MEDRAD® Intego PET Infusion System, or the MEDRAD® Centargo CT Injection System, all of which are provided by Bayer Healthcare LLC.

In some non-limiting embodiments, workstation device 106 may include one or more devices capable of being in communication with injection protocol management system 102, fluid injection system 104, and/or hospital information system 108 via communication network 110. For example, workstation device 106 may include a computing device, such as one or more computers, including a desktop computer, a laptop, tablet, and/or the like. At least a portion of the processes executed at the workstation device 106 may be executed at a remote server (e.g., a cloud computing server). In some non-limiting embodiments, workstation device 106 may provide a user interface for controlling operation of fluid injection system 104, including to generate instructions for and/or provide instructions to fluid injection system 104. Additionally or alternatively, workstation device 106 may display operational parameters of fluid injection system 104 during operation (e.g., during real-time operation) of fluid injection system 104. In some non-limiting embodiments, workstation device 106 may provide interconnectivity between fluid injection system 104 and other devices or systems, such as a scanner device (not shown). In some non-limiting embodiments, workstation device 106 may include the Certegra® Workstation provided by Bayer. In some non-limiting embodiments, workstation device 106 may include a display unit (e.g., a display device, a display screen, etc.), such as a computer monitor, a touchscreen, a heads-up display, and/or the like, which may be used to display a user interface (e.g., a graphical user interface (GUI) of a software application), via which a user may interact with workstation device 106 to view parameters and/or control operation of fluid injection system 104. For example, a user of workstation device 106 may provide inputs to workstation device 106 using one or more hardware or software components of the workstation device 106 in connection with a touch screen, a mouse, a trackpad, a keyboard, a stylus, a gesture-sensing camera, a microphone for receiving voice commands, and/or the like.

In some non-limiting embodiments, hospital information system 108 may include one or more devices capable of being in communication with injection protocol management system 102, fluid injection system 104, and/or workstation device 106 via communication network 110. For example, hospital information system 108 may include one or more computing devices, such as one or more desktop computers, one or more mobile devices, one or more servers, and/or the like. In some non-limiting embodiments, hospital information system 108 may include one or more subsystems, such as a patient procedure tracking system (e.g., a system that operates a modality worklist, a system that provides patient demographic information for fluid injection procedures and/or medical imaging procedures, etc.), a fluid injection management system, an image archive and communication system (e.g., a picture archive and communication system (PACS)), a radiology information system (RIS), and/or a radiology analytics system (e.g., the Radimetrics® Enterprise Application marketed and sold by Bayer HealthCare LLC). In some non-limiting embodiments, hospital information system 108 may include one or more databases that stores patient records (e.g., data files or data objects that include patient data) therein, which may be accessed (e.g., with read-access permission and/or write-access permission) by devices or systems in the environment 100A, such as injection protocol management system 102 and/or workstation device 106.

In some non-limiting embodiments, communication network 110 may include one or more wired and/or wireless networks. For example, communication network 110 may include a cellular network (e.g., a long-term evolution (LTE®) network, a third generation (3G) network, a fourth generation (4G) network, a fifth generation (5G) network, a sixth generation (6G) network, a code division multiple access (CDMA) network, etc.), a public land mobile network (PLMN), a local area network (LAN), a wide area network (WAN), a metropolitan area network (MAN), a telephone network (e.g., the public switched telephone network (PSTN)), a private network, an ad hoc network, an intranet, the Internet, a fiber optic-based network, a cloud computing network, a short range wireless communication network (e.g., a Bluetooth® network, a near field communication (NFC) network, etc.) and/or the like, and/or a combination of these or other types of networks.

The number and arrangement of systems and/or devices shown in FIG. 1A are provided as an example. There may be additional systems and/or devices, fewer systems and/or devices, different systems and/or devices, or differently arranged systems and/or devices than those shown in FIG. 1A. Furthermore, two or more systems and/or devices shown in FIG. 1A may be implemented within a single system or a single device, or a single system or a single device shown in FIG. 1A may be implemented as multiple, distributed systems or devices. Additionally or alternatively, a set of systems or a set of devices (e.g., one or more systems, one or more devices) of environment 100A may perform one or more functions described as being performed by another set of systems or another set of devices of environment 100A.

Referring now to FIG. 1B, FIG. 1B is a diagram of a non-limiting embodiment of system 100B for automatically generating an injection protocol for delivery of a medical fluid to a patient during a fluid injection procedure. In some non-limiting embodiments, one or more of the functions described herein with respect to system 100B may be performed (e.g., completely, partially, and/or the like) by injection protocol management system 102. In some non-limiting embodiments, one or more of the functions described with respect to system 100B may be performed (e.g., completely, partially, and/or the like) by another device or a group of devices separate from and/or including injection protocol management system 102, such as fluid injection system 104, workstation device 106 (e.g., which includes display unit 106A), hospital information system 108, and/or medical imaging system 112.

As shown in FIG. 1B, system 100B includes injection protocol management system 102, fluid injection system 104, workstation device 106, which includes display unit 106A, hospital information system 108, and medical imaging system 112. In some non-limiting embodiments, injection protocol management system 102 may interconnect (e.g., establish a connection to communicate with and/or the like) fluid injection system 104, workstation device 106, hospital information system 108, and/or medical imaging system 112 via wired connections, wireless connections, or a combination of wired and wireless connections.

In some non-limiting embodiments, medical imaging system 112 may include one or more devices capable of being in communication with injection protocol management system 102, fluid injection system 104, workstation device 106, and/or hospital information system 108 via a communication network (e.g., communication network 114). In some non-limiting embodiments, medical imaging system 112 may include one or more scanners, such as a CT scanner and/or an MRI scanner, capable of communicating via a communication network and capable of performing medical imaging procedures involving the use of a radiological contrast material.

As shown in FIG. 1B, injection protocol management system 102 may include a plurality of applications, such as informatics application 202A, data management application 202B, and imaging system connectivity application 202C. In some non-limiting embodiments, the plurality of applications may be stored in a memory device of injection protocol management system 102. In some non-limiting embodiments, each of the plurality of applications may be associated with an API associated with a respective application (e.g., a first API associated with a first application, a second API associated with a second application, a third API associated with a third application, etc.) that allows fluid injection system 104 to interface (e.g., communicate, establish a communication interface, etc.) with injection protocol management system 102 and/or that allows injection protocol management system 102 to interface with hospital information system 108 (e.g., individual subsystems of hospital information system 108, such as patient procedure tracking system 208A, fluid injector management system 208B, image archive and communication system 208C, radiology information system 208D, and/or radiology analytics system 208E).

In some non-limiting embodiments, injection protocol management system 102 may provide a user interface (e.g., via an application that includes a user interface, such as a web-based user interface) that allows a user to control fluid injection system 104. For example, a user may be able to initiate medical imaging, fluid injection, and/or other additional procedures to be performed by fluid injection system 104 via the user interface. Additionally or alternatively, the user interface may allow the user to control aspects of informatics application 202A, data management application 202B, and/or imaging system connectivity application 202C.

As further shown in FIG. 1B, workstation device 106 may include display unit 106A. In some non-limiting embodiments, display unit 106A may be capable of displaying the user interface (e.g., the web-based user interface) provided by injection protocol management system 102. In some non-limiting embodiments, display unit 106A may include a computing device, such as a smart display unit, a portable computer, such as a tablet, a laptop, and/or the like. In some non-limiting embodiments, display unit 106A may include a touchscreen for receiving inputs by a user. In some non-limiting embodiments, display unit 106A may include a display device (e.g., a monitor, a screen, and/or the like for displaying visual information).

In some non-limiting embodiments, informatics application 202A, data management application 202B, and/or imaging system connectivity application 202C may be associated with a communication interface of injection protocol management system 102 between fluid injection system 104 and hospital information system 108. For example, injection protocol management system 102 may provide a communication interface between fluid injection system 104 and hospital information system 108 based on informatics application 202A, data management application 202B, and/or imaging system connectivity application 202C. In some non-limiting embodiments, a communication interface may include a set of communication settings (e.g., protocols, standards, etc.) that enables one device (e.g., one device of a system) to communicate (e.g., telecommunicate) with another device. In some non-limiting embodiments, informatics application 202A may provide functionality for fluid injection system 104 to interact with hospital information system 108 (e.g., any of the plurality of subsystems of hospital information system 108). Additionally or alternatively, informatics application 202A may provide for storage of data for fluid injection system 104 (e.g., data associated with an injection protocol for a fluid injection procedure to be executed by fluid injection system 104). In some non-limiting embodiments, data management application 202B may provide functionality associated with file system and database management for injection protocol management system 102. Additionally or alternatively, data management application 202B may provide for operational control regarding storage of data for fluid injection system 104. In some non-limiting embodiments, imaging system connectivity application 202C may provide functionality for injection protocol management system 102 and/or fluid injection system 104 to interact with medical imaging system 112. In some non-limiting embodiments, imaging system connectivity application 202C may abstract an interface of medical imaging system 112 (e.g., an ISI interface, an ISI2 interface, and/or a Connect CT interface) to a standard format for injection protocol management system 102 and/or fluid injection system 104 to utilize.

In some non-limiting embodiments, injection protocol management system 102 may transmit data associated with an image received from medical imaging system 112 to fluid injection system 104 via a communication network. For example, injection protocol management system 102 may transmit data associated with the image received from medical imaging system 112 to fluid injection system 104 via the communication network based on an API call to imaging system connectivity application 202C from fluid injection system 104. In some non-limiting embodiments, injection protocol management system 102 may transmit data associated with a fluid injection procedure (e.g., data associated with an amount of time for injecting a radiological contrast material into a patient) received from fluid injection system 104 to medical imaging system 112 via the communication network. For example, injection protocol management system 102 may transmit the data associated with the fluid injection procedure received from fluid injection system 104 to medical imaging system 112 via the communication network based on an API call (e.g., an API call for an imaging system interface (ISI), an API call for an ISI2 interface, an API call for a Connect CT interface, etc.) to imaging system connectivity application 202C from medical imaging system 112. In some non-limiting embodiments, medical imaging system 112 may perform a medical imaging procedure on a patient based on the data, inclusive of an injection protocol, associated with the fluid injection procedure. In some non-limiting embodiments, injection protocol management system 102 may receive data associated with an operation of medical imaging system 112 from medical imaging system 112 via the communication network based on an API call (e.g., an API call from injection protocol management system 102 to medical imaging system 112).

In some non-limiting embodiments, injection protocol management system 102 may provide a communication interface between hospital information system 108 and fluid injection system 104 such that informatics application 202A provides the ability for fluid injection system 104 to receive data based on an API call from fluid injection system 104 to injection protocol management system 102. In some non-limiting embodiments, injection protocol management system 102 may transmit data associated with informatics received from hospital information system 108 to fluid injection system 104 via a communication network (e.g., communication network 110). For example, injection protocol management system 102 may transmit data associated with informatics received from hospital information system 108 to fluid injection system 104 via the communication network based on an API call to informatics application 202A from fluid injection system 104. Data associated with informatics may include data associated with identification of a patient, data associated with a patient examination procedure (e.g., a fluid injection procedure and/or a medical imaging procedure performed on a patient), such as data associated with a contrast fluid provided during a fluid injection procedure, a gauge of a catheter used during a fluid injection procedure, and a fluid injection protocol for a fluid injection procedure. In some non-limiting embodiments, injection protocol management system 102 may store data associated with a patient examination procedure and/or data associated with a configuration of fluid injection system 104 with informatics application 202A.

As further shown in FIG. 1B, hospital information system 108 may include a plurality of subsystems. The plurality of subsystems may include patient procedure tracking system 208A, fluid injector management system 208B, image archive and communication system 208C, radiology information system 208D, and radiology analytics system 208E. In some non-limiting embodiments, injection protocol management system 102 may receive the data associated with informatics from hospital information system 108 via a communication network (e.g., communication network 110), according to a communications protocol for communicating the data associated with informatics. For example, injection protocol management system 102 may receive data associated with a patient procedure from hospital information system 108 (e.g., from patient procedure tracking system 208A) via the communication network according to a Digital Imaging and Communications in Medicine (DICOM) communications protocol, data associated with an operation of the fluid injection system from hospital information system 108 (e.g., from fluid injector management system 208B) via the communication network based on an API call (e.g., an API call from injection protocol management system 102 to fluid injector management system 208B), data associated with a radiology image from hospital information system 108 (e.g., from image archive and communication system 208C) via the communication network according to a DICOM communications protocol, data associated with a patient examination procedure from hospital information system 108 (e.g., from radiology information system 208D) via the communication network according to a Health Level Seven (HL7) standard communications protocol, and/or data associated with radiation dosage during a medical imaging procedure from hospital information system 108 (e.g., from radiology analytics system 208E) via the communication network based on an API call (e.g., an API call from injection protocol management system 102 to radiology analytics system 208E).

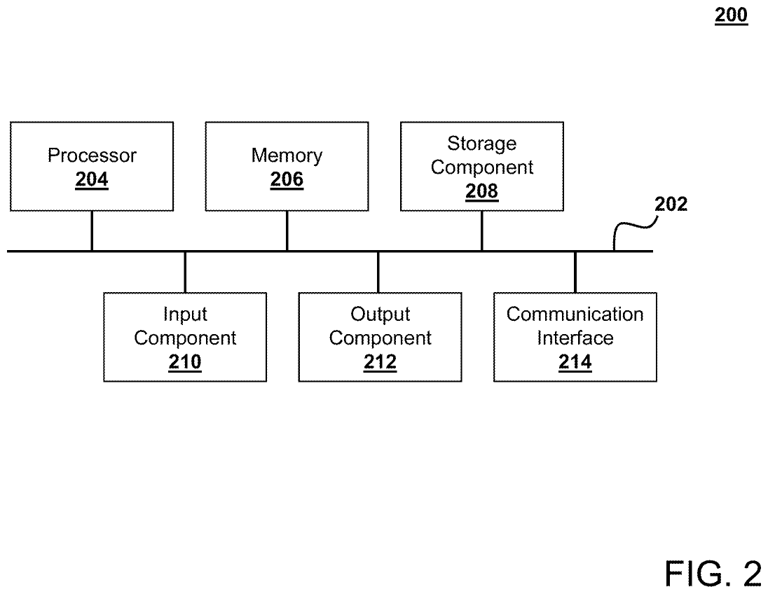

Referring now to FIG. 2, FIG. 2 is a diagram of example components of a device 200. Device 200 may correspond to one or more devices of injection protocol management system 102, one or more devices of fluid injection system 104, and/or workstation device 106, hospital information system 108, and/or medical imaging system 112. In some non-limiting embodiments, injection protocol management system 102, fluid injection system 104, and/or workstation device 106, hospital information system 108, and/or medical imaging system 112 may include at least one device 200 and/or at least one component of device 200. As shown in FIG. 2, device 200 may include bus 202, processor 204, memory 206, storage component 208, input component 210, output component 212, and communication interface 214.

Bus 202 may include a component that permits communication among the components of device 200. In some non-limiting embodiments, processor 204 may be implemented in hardware, firmware, or a combination of hardware and software. For example, processor 204 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 206 may include random access memory (RAM), read only memory (ROM), and/or another type of dynamic or static storage device (e.g., flash memory, magnetic memory, optical memory, etc.) that stores information and/or instructions for use by processor 204.

Storage component 208 may store information and/or software related to the operation and use of device 200. For example, storage component 208 may include a hard disk (e.g., a magnetic disk, an optical disk, a magneto-optic disk, a solid state disk, etc.), a compact disc (CD), a digital versatile disc (DVD), a floppy disk, a cartridge, a magnetic tape, and/or another type of computer-readable medium, along with a corresponding drive.

Input component 210 may include a component that permits device 200 to receive information, such as via user input (e.g., a touch screen display, a keyboard, a keypad, a mouse, a button, a switch, a microphone, etc.). Additionally, or alternatively, input component 210 may include a sensor for sensing information (e.g., a global positioning system (GPS) component, an accelerometer, a gyroscope, an actuator, etc.). Output component 212 may include a component that provides output information from device 200 (e.g., a display, a speaker, one or more light-emitting diodes (LEDs), etc.).

Communication interface 214 may include a transceiver-like component (e.g., a transceiver, a separate receiver and transmitter, etc.) that enables device 200 to communicate with other devices, such as via a wired connection, a wireless connection, or a combination of wired and wireless connections. Communication interface 214 may permit device 200 to receive information from another device and/or provide information to another device. For example, communication interface 214 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 200 may perform one or more processes described herein. Device 200 may perform these processes based on processor 204 executing software instructions stored by a computer-readable medium, such as memory 206 and/or storage component 208. A computer-readable medium (e.g., a non-transitory computer-readable medium) is defined herein as a non-transitory memory device. A memory device may include memory space located inside of a single physical storage device or memory space spread across multiple physical storage devices.

Software instructions may be read into memory 206 and/or storage component 208 from another computer-readable medium or from another device via communication interface 214. When executed, software instructions stored in memory 206 and/or storage component 208 may cause processor 204 to perform one or more processes described herein. Additionally, or alternatively, hardwired circuitry may be used in place of or in combination with software instructions to perform one or more processes described herein. Thus, embodiments described herein are not limited to any specific combination of hardware circuitry and software.

The number and arrangement of components shown in FIG. 2 are provided as an example. In some non-limiting embodiments, device 200 may include additional components, fewer components, different components, or differently arranged components than those shown in FIG. 2. Additionally, or alternatively, a set of components (e.g., one or more components) of device 200 may perform one or more functions described as being performed by another set of components of device 200.

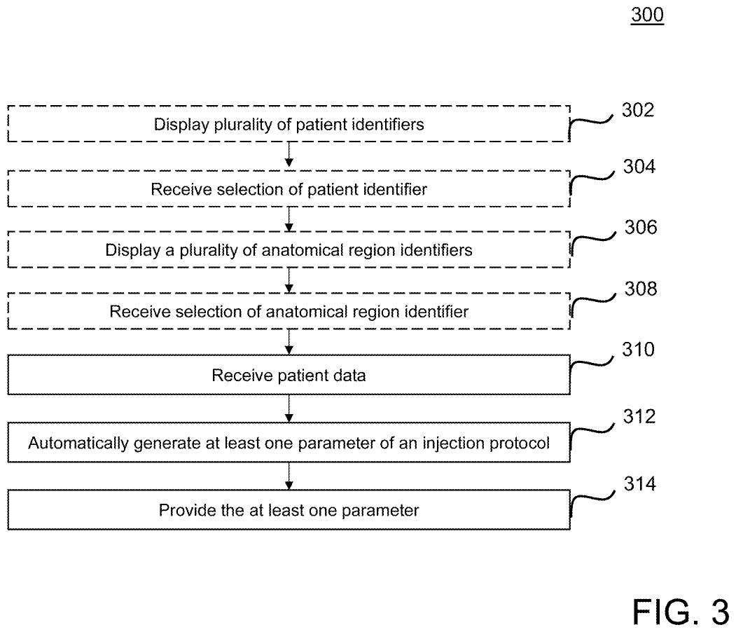

Referring now to FIG. 3, FIG. 3 is a flowchart of a non-limiting embodiment of a process 300 for operating a fluid injection system. In some non-limiting embodiments, one or more of the steps of process 300 are performed (e.g., completely, partially, etc.) by injection protocol management system 102. In some non-limiting embodiments, one or more of the steps of process 300 are performed (e.g., completely, partially, etc.) by another device or a group of devices separate from or including injection protocol management system 102, such as fluid injection system 104 (e.g., one or more devices of fluid injection system 104), workstation device 106, and/or hospital information system 108 (e.g., one or more devices of hospital information system 108).

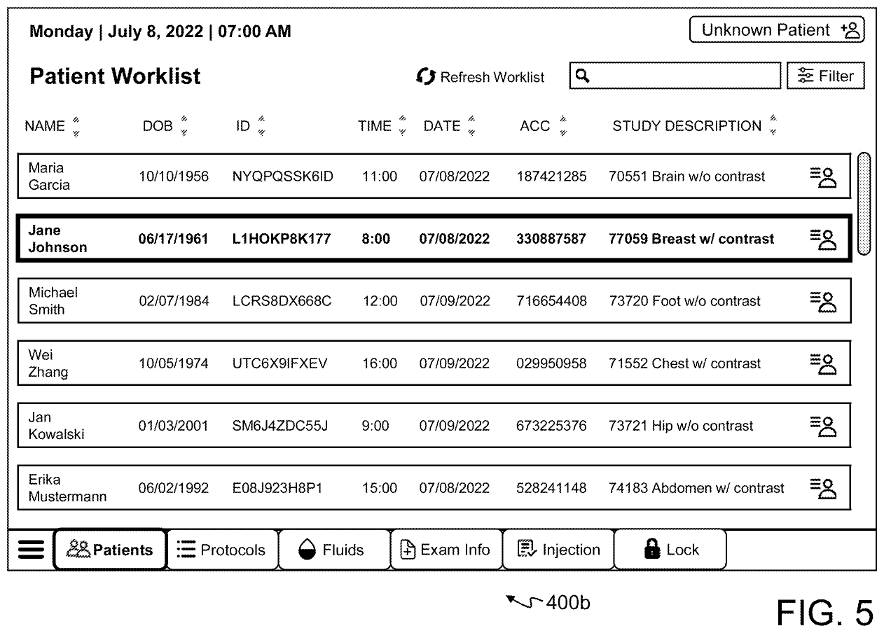

As shown in FIG. 3, at step 302, process 300 may include displaying a plurality of patient identifiers. For example, injection protocol management system 102 may cause a plurality of patient identifiers to be displayed in a user interface on a display unit (e.g., display unit 106A) of workstation device 106. In some non-limiting embodiments, the plurality of patient identifiers may include one or more unique identifiers. Each identifier may be associated with a patient record of a patient (e.g., a patient record of a patient stored in patient procedure tracking system 208A of hospital information system 108). In some non-limiting embodiments, the plurality of patient identifiers may be displayed in a list of rows associated with patients (e.g., as shown in FIG. 5). In some non-limiting embodiments, the plurality of patient identifiers may include an identifier that is not associated with a patient record, such as an identifier for an unknown or anonymous patient. In some non-limiting embodiments, the plurality of patient identifiers may include one or more patient identifiers associated with a patient record and one or more identifiers that are not associated with a patient record.

As shown in FIG. 3, at step 304, process 300 may include receiving a selection of a patient identifier. For example, injection protocol management system 102 may receive a selection of a patient identifier of a plurality of patient identifiers via a user interface. In some non-limiting embodiments, injection protocol management system 102 may receive the selection of a patient identifier based on user input. For example, a user may select an identifier (e.g., a patient identifier that includes a unique identifier) associated with a patient record by selecting (e.g., touching, clicking, pressing, etc.) the identifier in a user interface. In some non-limiting embodiments, the user may select an identifier that is not associated with a patient record by selecting a button indicating that the patient is unknown, anonymous, and/or the like. In the example above, injection protocol management system 102 may receive the selection of the identifier via the user interface based on the user selecting the identifier in the user interface.

As shown in FIG. 3, at step 306, process 300 may include displaying a plurality of anatomical region identifiers. For example, injection protocol management system 102 may cause a plurality of anatomical region identifiers to be displayed in a user interface on a display unit (e.g., display unit 106A of workstation device 106). In some non-limiting embodiments, an anatomical region identifier may include an identifier (e.g., a short description, a unique code, an image, etc.) that represents an anatomical region (e.g., an anatomical region of a patient) in which a fluid injection procedure is to take place. For example, a fluid injection procedure may entail injecting a medical fluid, such as a contrast agent, so that an MRI scan can be taken of a breast of the patient. In such an example, the anatomical region identifier may include a short description that is the text “Breast.” By way of further example, the anatomical region identifier may include a unique code (e.g., a cooperative magnetic imaging (CPT) code), such as “77059.”

As shown in FIG. 3, at step 308, process 300 may include receiving a selection of an anatomical region identifier. For example, injection protocol management system 102 may receive a selection of an anatomical region identifier of a plurality of anatomical region identifiers via a user interface. In some non-limiting embodiments, a user may select an anatomical region identifier by selecting the anatomical region identifier in a user interface. In some non-limiting embodiments, injection protocol management system 102 may receive the selection of an anatomical region identifier based on user input. For example, a user may select an anatomical region identifier associated with an anatomical region of a patient in which a fluid injection procedure is to take place by selecting the anatomical region identifier in the user interface. In such an example, injection protocol management system 102 may receive the selection of the anatomical region identifier via the user interface based on the user selecting the anatomical region identifier in the user interface. In some non-limiting embodiments, the selection of an anatomical region identifier may be performed automatically via a trigger electronically communicated from, for example, workstation device 106 by virtue of the selection of the patient on workstation device 106.

As shown in FIG. 3, at step 310, process 300 includes receiving patient data. For example, injection protocol management system 102 may receive patient data associated with a patient undergoing a fluid injection procedure. In some non-limiting embodiments, patient data may include a name of a patient, a height of a patient, a weight of a patient, an age of a patient, an estimated glomerular filtration rate (eGFR) of a patient, a sex of a patient, a patient identifier (e.g., social security number, global unique code, etc.), a patient accession number (e.g., a unique number assigned to the patient by hospital information system 108 that may be used to determine when the patient was first identified), a patient procedure identifier (e.g., a unique identifier to identify a type of procedure that is to take place, such as a medical procedure, including a fluid injection procedure), and/or the like. In some non-limiting embodiments, patient data associated with a patient may correspond to a patient record for the patient which is stored (e.g., an existing patient record) in hospital information system 108.

In some non-limiting embodiments, injection protocol management system 102 may transmit an identifier (e.g., a unique identifier) associated with a patient record for a patient undergoing a fluid injection procedure to hospital information system 108 and injection protocol management system 102 may receive the patient data from hospital information system 108. In some non-limiting embodiments, hospital information system 108 may receive the identifier associated with the patient record, retrieve the patient record for the patient based on the identifier, and transmit the patient record to injection protocol management system 102.

In some non-limiting embodiments, injection protocol management system 102 may receive patient data based on a selection of a patient identifier. In some non-limiting embodiments, injection protocol management system 102 may receive the patient data by retrieving the patient data from a patient record that is stored in hospital information system 108 based on the patient identifier. In some non-limiting embodiments, injection protocol management system 102 may retrieve the patient data from hospital information system 108 and store the patient data in temporary local storage on injection protocol management system 102 based on receiving the selection of the patient identifier. In some non-limiting embodiments, injection protocol management system 102 may cause the patient data to be displayed in a user interface. In some non-limiting embodiments, when the patient identifier that is selected is an identifier that is unassociated with a patient record, injection protocol management system 102 may receive the patient data from user input via the user interface. For example, injection protocol management system 102 may cause one or more input fields (e.g., graphical user interface elements displayed in a user interface, such as text fields, number sliders, dropdown menus, date pickers, etc.) to be displayed to receive user input that includes the patient data (e.g., by receiving a weight in pounds when the user types in a patient weight into a corresponding patient weight input field for the unknown patient).

As shown in FIG. 3, at step 312, process 300 includes automatically generating at least one parameter of an injection protocol. For example, injection protocol management system 102 may automatically generate at least one parameter of an injection protocol for a fluid injection procedure based on patient data associated with a patient that is undergoing the fluid injection procedure. In some non-limiting embodiments, injection protocol management system 102 may automatically generate the at least one parameter of the injection protocol by determining a dosage (e.g., ml/kg) of a medical fluid (e.g., contrast fluid) for the fluid injection procedure. In some non-limiting embodiments, injection protocol management system 102 may determine the dosage based on a weight of the patient (e.g., in kilograms, which may be converted from pounds), a concentration of a medical fluid (e.g., mmol/ml) to be used in the fluid injection procedure, an anatomical region (e.g., breast, cardiac, central nervous system (CNS), renal supra-aorta, liver, head, neck, chest, abdomen, extremities, etc.) of the patient, and/or the like.

In some non-limiting embodiments, a fluid injection procedure may include a plurality of phases. For example, the fluid injection procedure may include an injection phase, a flush phase, and/or a hold phase. In some non-limiting embodiments, an injection phase of a fluid injection procedure may be a stage of the fluid injection procedure characterized by a volume of a medical fluid being injected at a flow rate (e.g., volume of fluid over time), which may be determined by an overall duration of the injection phase. In some non-limiting embodiments, a flush phase of a fluid injection procedure may be a stage of the fluid injection procedure characterized by a volume of a medical fluid that acts as a flushing agent being injected at a flow rate, which may be determined by an overall duration of the flush phase. In some non-limiting embodiments, a hold phase of a fluid injection procedure may be a stage of a fluid injection procedure characterized by no fluid being injected into a patient for a duration of time. In some non-limiting embodiments, injection protocol management system 102 may determine a dosage of a medical fluid for an injection phase and/or a flush phase of the fluid injection procedure. In some non-limiting embodiments, operational parameters of injection protocols for fluid injection procedures carried out by fluid injection system 104 may include one or more exemplary data types that are disclosed in U.S. patent application Ser. No. 10/143,562, filed on May 10, 2002, issued as U.S. Pat. No. 7,457,804; U.S. patent application Ser. No. 12/254,318, filed on Oct. 20, 2008, issued as U.S. Pat. No. 7,996,381; U.S. patent application Ser. No. 13/180,175, filed on Jul. 11, 2011, issued as U.S. Pat. No. 8,521,716, the disclosures of each of which are incorporated herein by reference in their entireties.

In some non-limiting embodiments, a fluid injection procedure may include an injection phase, a flush phase, and/or a hold phase in any order as appropriate. In some non-limiting embodiments, a parameter of an injection protocol for a fluid injection procedure may include an amount of time (e.g., for each phase) of the fluid injection procedure, a type of medical fluid (e.g., for each phase) of the fluid injection procedure, a concentration of a type of medical fluid (e.g., for each phase) of the fluid injection procedure, a dosage of a medical fluid (e.g., for each phase) of the fluid injection procedure, a flow rate of a medical fluid (e.g., for each phase) of the fluid injection procedure, and/or the like.

In some non-limiting embodiments, injection protocol management system 102 may determine the at least one parameter of an injection protocol for a fluid injection procedure. For example, injection protocol management system 102 may retrieve data associated with the at least one parameter from a data structure based on data associated with the fluid injection procedure (e.g., patient data associated with a patient that is to undergo the fluid injection procedure, data associated with an anatomical region of a patient in which the fluid injection procedure is to take place, etc.). In some non-limiting embodiments, injection protocol management system 102 may configure the at least one parameter of an injection protocol for a fluid injection procedure. For example, injection protocol management system 102 may configure fluid injection system 104 (e.g., a fluid injection device of fluid injection system 104) so that the fluid injection procedure is carried out according to the at least one parameter of the injection protocol.

In some non-limiting embodiments, a fluid injection procedure may include a plurality of each of an injection phase, a flush phase, and/or a hold phase. For example, a fluid injection procedure may include two injection phases. In some non-limiting embodiments, injection protocol management system 102 may determine a first flow rate of a medical fluid for a first injection phase and a second flow rate of the medical fluid for a second injection phase. For example, injection protocol management system 102 may determine the first flow rate and/or the second flow rate based on a concentration of the medical fluid (e.g., a mixture/ratio of contrast agent to saline) and/or an anatomical region of a patient in which a fluid injection procedure is to take place.

In some non-limiting embodiments, injection protocol management system 102 may automatically generate the at least one parameter of the injection protocol based on an anatomical region identifier. In one example, injection protocol management system 102 may automatically generate the at least one parameter of the injection protocol based on receiving a selection of the anatomical region identifier.

In another example, injection protocol management system 102 may automatically generate an injection protocol for a fluid injection procedure having two phases, such as an injection phase and a flush phase, and injection protocol management system 102 may determine a type of a medical fluid and a dosage of the medical fluid for the injection phase based on a weight of a patient and an anatomical region identifier.

In yet another example, injection protocol management system 102 may automatically generate parameters of an injection protocol by determining a first dosage of a medical fluid, determining parameters for a first flush phase, determining a second dosage of the medical fluid, and determining parameters for a second flush phase. In some non-limiting embodiments, injection protocol management system 102 may determine a first dosage of a medical fluid, configure a first flush phase (e.g., by configuring a volume of saline fluid and flow rate of the saline fluid to be used by fluid injection system 104) of the fluid injection procedure including a first injection of a saline fluid. Injection protocol management system 102 may further determine a second dosage of the medical fluid for a second injection phase of the fluid injection procedure based on at least one of the following: the weight of the patient, the concentration of the medical fluid, or the anatomical region. Injection protocol management system 102 may further configure a second flush phase (e.g., by configuring a volume of a same or different saline fluid and a flow rate of the same or different saline fluid) including a second injection of the saline fluid. Injection protocol management system 102 may be configured to execute the fluid injection procedure (e.g., by communicating instructions to fluid injection system 104), in order of the first injection phase, the first flush phase, a hold phase, the second injection phase, and the second flush phase. For example, such a configuration may be used for a fluid injection procedure accompanying an MRI scan of a cardiac region of a patient.

In some non-limiting embodiments, injection protocol management system 102 may automatically generate the at least one parameter of the injection protocol for the fluid injection procedure based on patient data of a patient and independent of (e.g., without) an input from a user associated with the at least one parameter. For example, injection protocol management system 102 may automatically generate at least one parameter of an injection protocol for the fluid injection procedure based on the patient data and independent of an input from a user associated with a dosage of a medical fluid for the fluid injection procedure received via a user interface associated with a fluid injection system that is to execute the fluid injection procedure.

As shown in FIG. 3, at step 314, process 300 includes providing the at least one parameter. For example, injection protocol management system 102 may provide the at least one parameter of the injection protocol to fluid injection system 104 (e.g., a fluid injection device of fluid injection system 104). In some non-limiting embodiments, the at least one parameter of the injection protocol may be provided to the fluid injection system 104 for execution of the fluid injection procedure. In one example, fluid injection system 104 may cause a medical fluid to be injected into a patient via a fluid injection device as part of a fluid injection procedure based on receiving data associated with the at least one parameter of the injection protocol from injection protocol management system 102. Fluid injection system 104 may execute the fluid injection procedure according to the at least one parameter of the injection protocol, such as a dosage of the medical fluid. In some non-limiting embodiments, injection protocol management system 102 may automatically provide the at least one parameter to fluid injection system 104 based on a user input (e.g., a selection of a start button). In some non-limiting embodiments, injection protocol management system 102 may provide the at least one parameter in a user interface to be displayed as an indication that the fluid injection procedure is to take place before the fluid injection procedure is initiated. In some non-limiting embodiments, injection protocol management system 102 may cause fluid injection system 104 to execute the fluid injection procedure. For example, injection protocol management system 102 may cause fluid injection system 104 to execute the fluid injection procedure based on injection protocol management system 102 receiving a user input (e.g., a selection of a start button).

In some non-limiting embodiments, injection protocol management system 102 may cause a fluid injection procedure to be executed by fluid injection system 104. For example, injection protocol management system 102 may transmit a command signal that causes the fluid injection procedure to be executed by fluid injection system 104 (e.g., based on fluid injection system 104 receiving the command signal) according to an injection protocol of the fluid injection procedure. In some non-limiting embodiments, injection protocol management system 102 may cause a fluid injection procedure to be executed in order of a plurality of phases. For example, injection protocol management system 102 may cause a fluid injection procedure to be executed in order of an injection phase, a flush phase, and a hold phase, or any combination as appropriate. In one example, injection protocol management system 102 may cause a fluid injection procedure to be executed in order of a first injection phase, a first flush phase, a hold phase, a second injection phase, a second flush phase.

In some non-limiting embodiments, injection protocol management system 102 may transmit data associated with the fluid injection procedure (e.g., data associated with a plurality of parameters of an injection protocol of the fluid injection procedure, data associated with at least one parameter of an injection protocol of the fluid injection procedure, data associated with a complete injection protocol of the fluid injection procedure, etc.). For example, injection protocol management system 102 may transmit the data associated with the fluid injection procedure to fluid injection system 104 prior to execution of the fluid injection procedure.

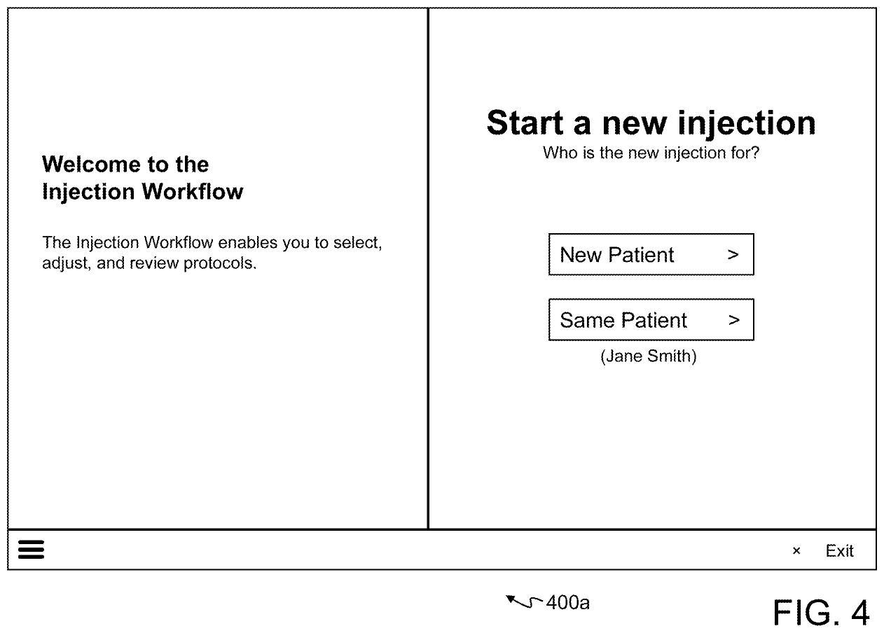

Referring now to FIG. 4, FIG. 4 illustrates a non-limiting embodiment of a first GUI 400a for use in the described methods for operating a fluid injection system. It will be appreciated that the precise style, arrangement, and configuration of interface components of the first GUI 400a are for exemplary purposes only and are not to be taken as limiting on the present disclosure. One or more elements of another GUI described herein may be combined with that of the first GUI 400a, and one or more elements of the first GUI 400a may be included in other GUIs, to provide the features of the described methods for operating a fluid injection system. While ordinal words like “first”, “second”, “third”, etc., may be used to describe the GUIs in connection with FIGS. 4 through 7E, it will be appreciated that these terms are to differentiate exemplary views (e.g., graphically displayed windows) rather than to prescribe a sequence of operation, unless specified otherwise. A user interface as described in the present disclosure may include one or more of the GUIs 400a-400i described herein.

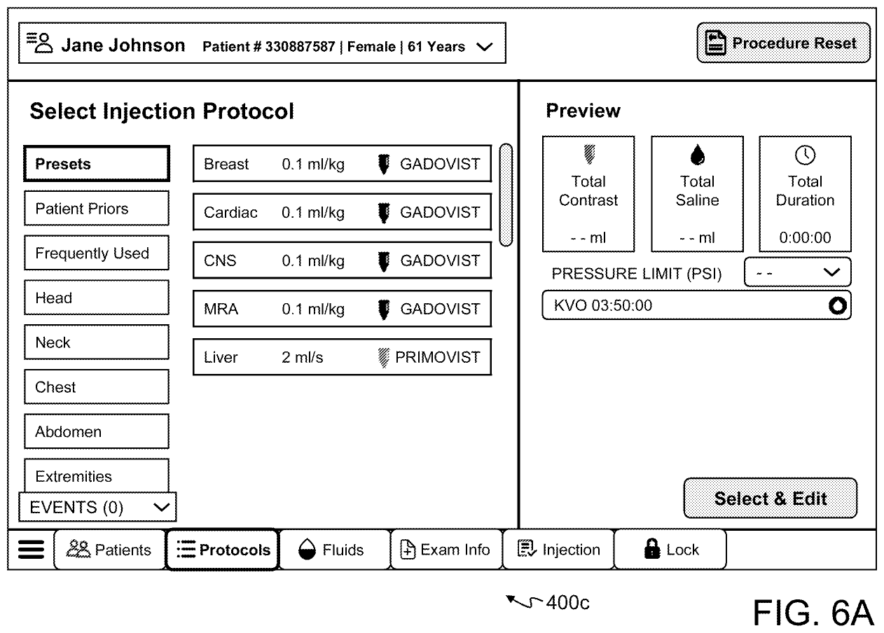

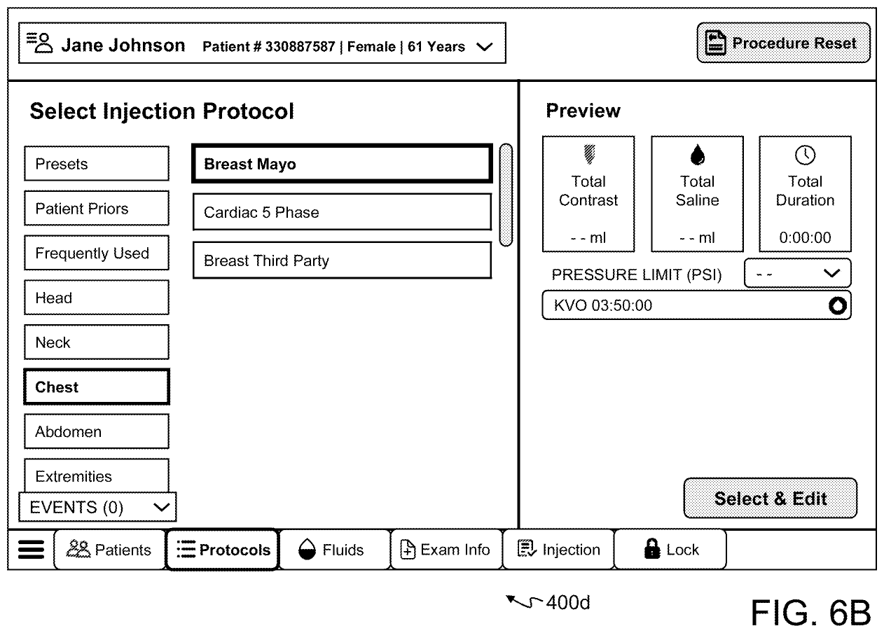

The first GUI 400a depicts an illustrative welcome screen (e.g., starting screen, home screen, etc.) for display to a user of the workstation device 106. The first GUI 400a may include a settings or menu icon (e.g., depicted as a cog symbol or a three-line symbol) to allow the user to configure preferences (e.g., units of systems of measurement, dosage calculator default setting, etc.) and/or display settings (e.g., brightness, light or dark mode, font size, etc.) for the GUIs of the described methods. The first GUI 400a may further include an exit control button (e.g., an “X” that may be accompanied by the word “exit”) to close out of the GUIs of the described methods. The first GUI 400a may further include a set of text describing the GUIs that will be displayed, such as a welcome message with information about getting started. The first GUI 400a may further include a graphical button for starting a fluid injection procedure with a new patient (e.g., a button labeled “New Patient”), and a graphical button for starting a fluid injection procedure with the last patient that the system was used for (e.g., a button labeled “Same Patient”). Selecting the new patient button may navigate the user to a second GUI 400b, as shown in FIG. 5. Selecting the same patient button may navigate the user to a third GUI 400c, as shown in FIG. 6A, but based on the patient that the system was previously used for.

Referring now to FIG. 5, FIG. 5 illustrates a non-limiting embodiment of a second GUI 400b for use in the described methods for operating a fluid injection system. It will be appreciated that the precise style, arrangement, and configuration of interface components of the second GUI 400b are for exemplary purposes only and are not to be taken as limiting on the present disclosure. One or more elements of another GUI described herein may be combined with that of the second GUI 400b, and one or more elements of the second GUI 400b may be included in other GUIs, to provide the features of the described methods for operating a fluid injection system.

The second GUI 400b depicts an illustrative patient worklist screen that provides the user with a list of patients (e.g., including at least a plurality of patient identifiers, such as unique identifiers associated with patient records). The second GUI 400b may provide a list of patients that have been scheduled for, at least in part, one or more fluid injection procedures. The workstation device 106 may communicate with the hospital information system 108 to determine the list of patients to display, based on the time (e.g., date, time of day, etc.) that a patient procedure is scheduled to take place. For example, workstation device 106 may receive a list of patients that are scheduled closest to the present time of operation of the workstation device 106. In the example as shown, for a user operating the workstation device 106 on Jul. 8, 2022, at 07:00 AM, the second GUI 400b may display a list of patients that have procedures scheduled over at least the following forty-eight hours (e.g., Jul. 8, 2022, and July 9, 2022). It will be appreciated that patients scheduled for wider or narrower time periods may be displayed in the second GUI 400b. It will also be appreciated that a particular patient may be shown more than once in the second GUI 400b if the patient is scheduled for more than one procedure.

In some non-limiting embodiments, the second GUI 400b may provide the list of patients in a series of rows, where each row represents a different patient and the patient's scheduled procedure. Each column of the depicted data may represent a different portion of patient data. For example, a plurality of columns may be provided that depict, without limitation, patient name (e.g., titled “NAME”), date of birth (e.g., titled “DOB”), patient identifier (e.g., titled “ID”), time of procedure (e.g., titled “TIME”), date of procedure (e.g., titled “DATE”), accession number (e.g., titled “ACC”, a unique number assigned to the patient by a registrar that may be used to determine when the patient was first identified by the registrar), and description of the procedure to be executed (e.g., titled “STUDY DESCRIPTION”, which may include a procedure identifier and/or a short text description of the procedure). The rows may be configured in the second GUI 400b to be sortable based on a selected column (e.g., when “NAME” is selected, the patients may be organized in alphabetical order of last name, in anti-alphabetical order of last name, and/or the like). Each row may further include an icon (e.g., an icon depicting a profile symbol) that a user can select to view more information about the patient and/or the patient's procedure.

In some non-limiting embodiments, the second GUI 400b may include a refresh button, which may update the list of patients with the most current data and based on the current time. Additionally or alternatively, the second GUI 400b may automatically refresh based on a predetermined time interval (e.g., once a minute, once every five minutes, etc.). The second GUI 400b may further include a search field, to allow the user to search for and find a specific patient that is scheduled for a procedure. The second GUI 400b may further include a filter control, to provide the user with one or more filters to reconfigure the displayed patient data. If the number of patients exceeds the amount of display space in the second GUI 400b, the second GUI 400b may include a scrollbar and/or be scrollable to view additional patients. The second GUI 400b may also include a button to allow the user to continue the method for a patient that is unidentified or unassociated with a patient record (e.g., an anonymous patient, an unknown patient, etc.). Said button may be associated with an identifier that is unassociated with a patient record.

In some non-limiting embodiments, the second GUI 400b may include a settings or menu icon (e.g., depicted as a cog symbol or a three-line symbol) to allow the user to configure preferences and/or display settings for the GUIs of the described methods. The second GUI 400b may further include a navigation bar to depict where in the process the user is (e.g., in which portion of the process for setting up a fluid injection procedure). The navigation bar may include tabs with text and/or icons to represent the parts of the fluid injection procedure setup process, which may include, but are not limited to: viewing the patient worklist (e.g., a “Patients” tab); viewing injection protocol presets (e.g., a “Protocols” tab); viewing patient data and parameters for an injection protocol (e.g., a “Fluids” tab); viewing the generated parameters for an injection protocol of the fluid injection procedure (e.g., an “Exam Info” tab); confirming and initiating a fluid injection procedure (e.g., an “Injection” tab); and waiting while the fluid injection procedure is in progress (e.g., a “Lock” tab, to indicate that the parameters of an injection protocol for the fluid injection procedure have been configured and provided to a fluid injection system 104 and cannot be changed while the procedure takes place).

Referring now to FIG. 6A, FIG. 6A illustrates a non-limiting embodiment of a third GUI 400c for use in the described methods for operating a fluid injection system. It will be appreciated that the precise style, arrangement, and configuration of interface components of the third GUI 400c are for exemplary purposes only and are not to be taken as limiting on the present disclosure. One or more elements of another GUI described herein may be combined with that of the third GUI 400c, and one or more elements of the third GUI 400c may be included in other GUIs, to provide the features of the described methods for operating a fluid injection system.

The third GUI 400c depicts an illustrative injection protocol selection screen, via which a user can view various types of presets of injection protocols (e.g., including at least a plurality of anatomical region identifiers). The arrangement of the displayed injection protocols may be predetermined according to a specific set of medical fluids that are planned for use in an associated fluid injection system. For example, a plurality of preset injection protocols may be displayed for fluid injection procedures of a number of anatomical regions, such as, but not limited to: breast, cardiac, central nervous system (CNS), renal supra-aorta (e.g., via magnetic resonance angiography (MRA)), and liver. While the word “preset” may be used to denote a predetermined or preferred injection protocol to be displayed, it will be appreciated that not all parameters of the injection protocol may be configured until later in the described method, when said parameters may be automatically generated by the workstation device 106 based on patient data of the patient undergoing the procedure. The preset injection protocol buttons may identify, in addition to corresponding anatomical regions, a dosage parameter of a medical fluid to be used (e.g., ml/kg) and an identifier of the medical fluid such as, for example, Gadovist and Primovist as shown in FIG. 6A.

In some non-limiting embodiments, the third GUI 400c may include a number of other injection protocol selection options besides those that are preset for the workstation device 106. Other sets of injection protocols may be presented in response to user selection of corresponding buttons, such as patient priors (e.g., to show prior injection protocols used for the patient based on the patient's patient record), frequently used (e.g., to show injection protocols commonly used at the workstation device 106), and other templates for injection protocols for various groups of anatomical regions (e.g., head, neck, chest, abdomen, extremities, etc.). The third GUI 400c may further allow the user to create their own preset injection protocol. The third GUI 400c may also include an events notification window accessible by a collapsible control button, which may present alerts or notifications to the user of the workstation device 106. The third GUI 400c may further display patient data for a selected patient (if applicable) in a dropdown field, which may provide some high-level patient data for ease of reference, including, but not limited to, patient name, patient accession number, patient sex, and patient age. In this manner, the user is consistently apprised of the patient information for which the fluid injection procedure will take place.

In some non-limiting embodiments, the third GUI 400c may include a procedure reset button, to abandon the setup process for the current fluid injection procedure and go back to a home and/or starting screen, such as the first GUI 400a of FIG. 4, the second GUI 400b of FIG. 5, or the third GUI 400c of FIG. 6A. A confirmation from the user may be required before resetting the procedure, to avoid scenarios where the user inadvertently selects the option to reset the procedure. The third GUI 400c may also include a preview of the fluid injection procedure based on the injection protocol selected and configured up to that point in the setup process. Since the exemplary third GUI 400c shows a number of preset injection protocols and none of them have been selected by a user yet, the injection protocol preview portion of the third GUI 400c is depicted as mostly empty/unpopulated. For example, the preview may include fields to show the total contrast volume, the total saline volume, and the total duration of the fluid injection procedure. Each of these fields in the third GUI 400c are shown as undefined or zero, since no injection protocol has been selected or configured yet. Similarly, a depicted dropdown for the maximum injection pressure limit for the injection protocol (e.g., in units of pounds per square inch (PSI)) is still undefined. In contrast, a duration for keep-vein-open (KVO) time is depicted based on the patient's anticipated medical procedure. Once a user selects an injection protocol for further setup, the user may select a button (e.g., a “Select & Edit” button) to view the parameters of the protocol and confirm the parameters before initiating the procedure. Additionally, like the second GUI 400b, the third GUI 400c may include a navigation bar to depict where the user is in the setup process.

Referring now to FIG. 6B, FIG. 6B illustrates a non-limiting embodiment of a fourth GUI 400d for use in the described methods for operating a fluid injection system. It will be appreciated that the precise style, arrangement, and configuration of interface components of the fourth GUI 400d are for exemplary purposes only and are not to be taken as limiting on the present disclosure. One or more elements of another GUI described herein may be combined with that of the fourth GUI 400d, and one or more elements of the fourth GUI 400d may be included in other GUIs, to provide the features of the described methods for operating a fluid injection system.

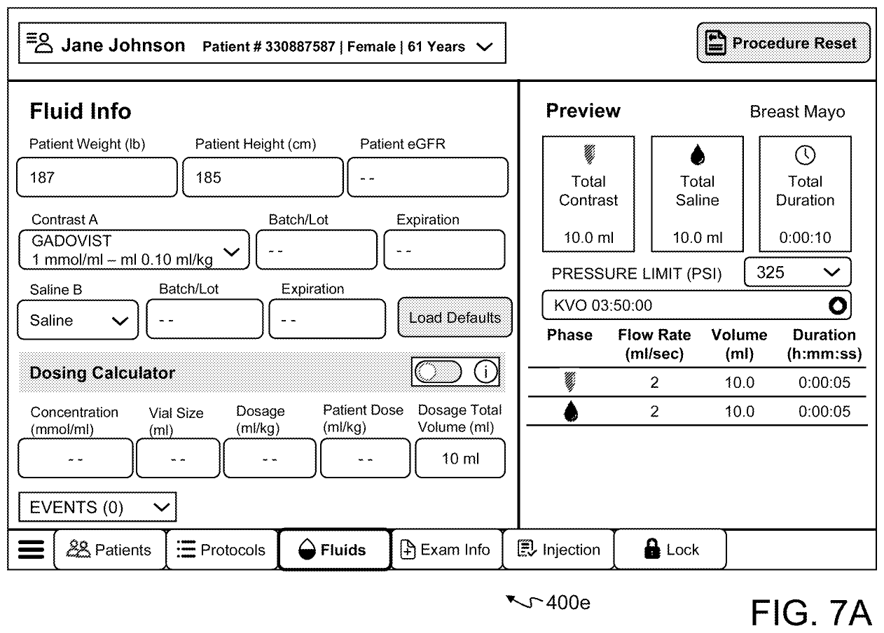

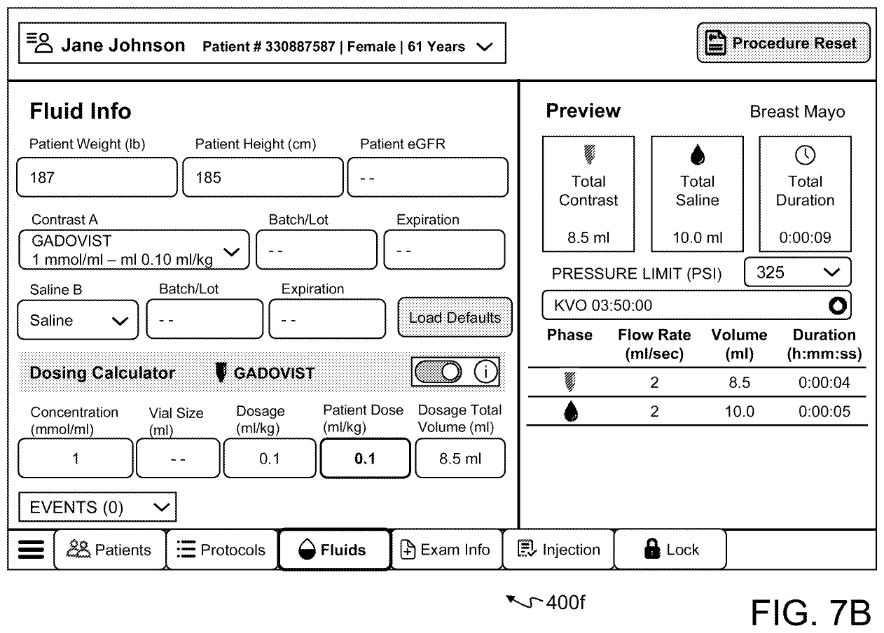

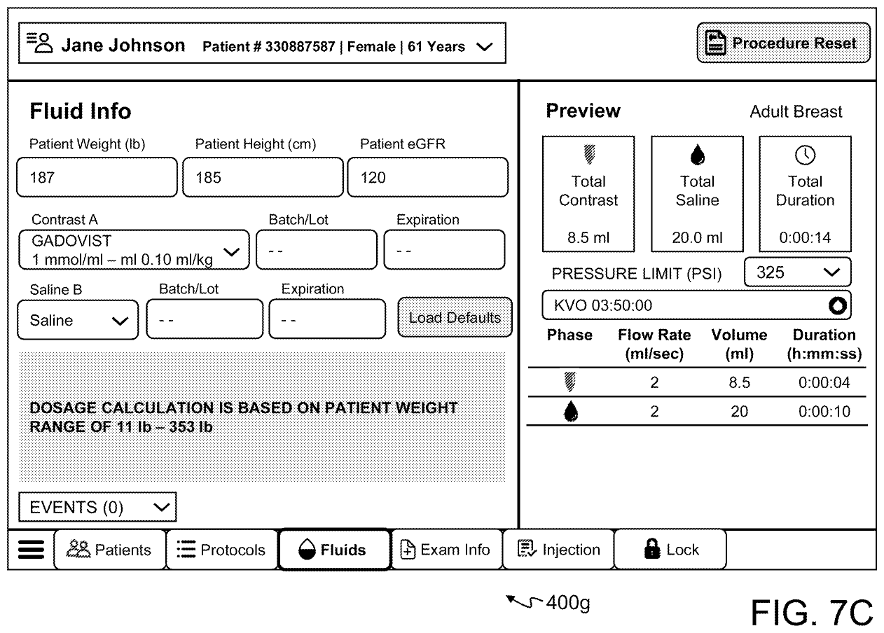

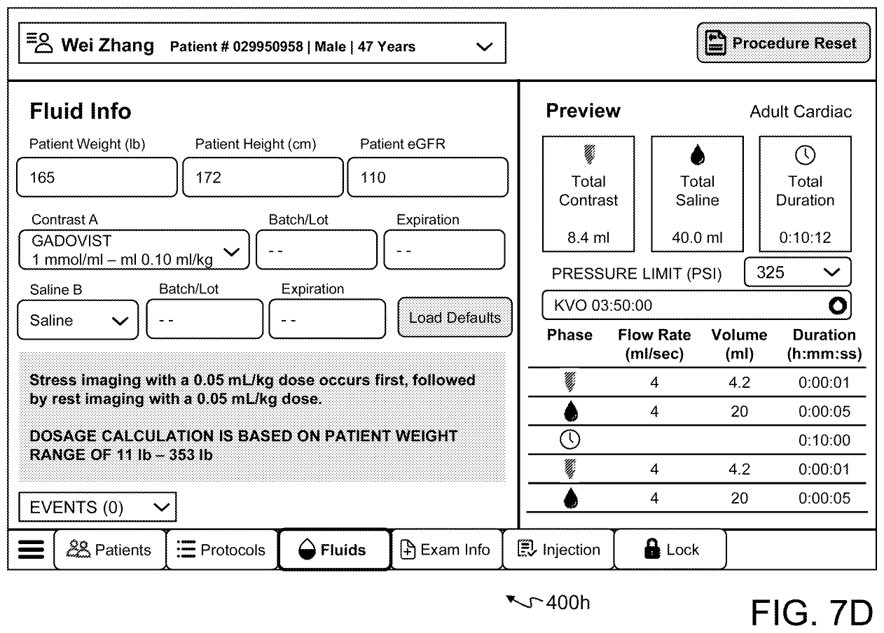

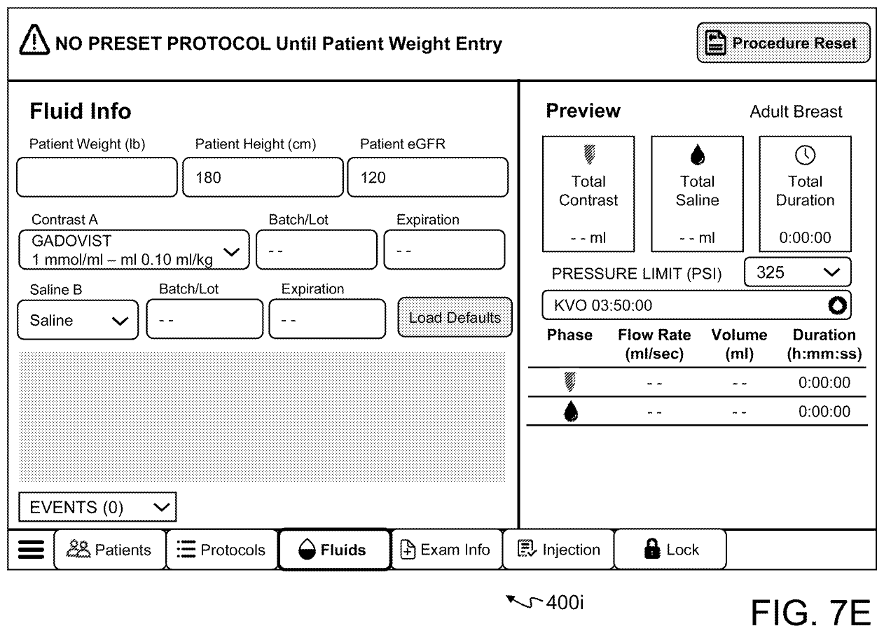

The fourth GUI 400d depicts substantially the same GUI as the third GUI 400c, but in a configuration where the user is viewing a different set of injection protocols. Instead of preset injection protocols being displayed, a user has selected a “Breast UAA Hospital” template for a breast-based injection protocol (e.g., based on guidelines from an exemplary hospital) that is listed under the injection protocols for the “Chest” grouping of injection protocols. Upon proceeding further (e.g., by selecting the “Select & Edit” button”), the user may view the specific parameters of the selected injection protocol (see FIG. 7A).

Referring now to FIG. 7A, FIG. 7A illustrates a non-limiting embodiment of a fifth GUI 400e for use in the described methods for operating a fluid injection system. It will be appreciated that the precise style, arrangement, and configuration of interface components of the fifth GUI 400e are for exemplary purposes only and are not to be taken as limiting on the present disclosure. One or more elements of another GUI described herein may be combined with that of the fifth GUI 400e, and one or more elements of the fifth GUI 400e may be included in other GUIs, to provide the features of the described methods for operating a fluid injection system.

The fifth GUI 400e depicts an illustrative window for viewing and modifying parameters for a selected injection protocol (e.g., titled “Fluids” or “Fluid Info”, in reference to the fluids being used in the fluid injection procedure). As shown, the example is for a selected “Breast UAA Hospital” template of injection protocol, which may have been selected by a user in the fourth GUI 400d of FIG. 6B. In some non-limiting embodiments, patient data may be retrieved from the hospital information system 108 and displayed in the fifth GUI 400e based on a selection of a patient identifier in the second GUI 400b. Additionally or alternatively, patient data may be pre-loaded from the hospital information system 108 and selectively displayed on the workstation device 106 based on the selection of a patient identifier. In the depicted example, patient data may be displayed in the fifth GUI 400e, including, but not limited to, patient weight, patient height, and patient eGFR. A patient information control (e.g., a dropdown), procedure reset button, event control, and/or navigation bar may continue to be displayed in the fifth GUI 400e, as they were in the third GUI 400c and fourth GUI 400d.

In some non-limiting embodiments, the window for viewing and modifying parameters for a selected injection protocol may include information about the fluids to be used in the fluid injection procedure. For example, the window may display a medical fluid selector (e.g., a dropdown depicting and allowing modification of a contrast fluid), a batch/lot field for the medical fluid, and an expiration field for the medical fluid. By way of further example, the window may display a saline fluid selector (e.g., a dropdown depicting and allowing modification of a saline fluid), a batch/lot field for the saline fluid, and an expiration field for the saline fluid. The window may also include a “load defaults” button, to load predetermined medical fluid and saline fluid data for the injection protocol.

The fifth GUI 400e of FIG. 7A, in particular, depicts an illustrative window where a dosing calculator feature is initially disabled. The dosing calculator of the present disclosure includes a tool to automatically determine medical fluid dosage for a patient based on patient data, without the need for users to consult external tools. Because the dosage calculator is disabled in the fifth GUI 400e, the injection protocol preview of the fifth GUI 400e is based off of default dosage volumes that are not necessarily customized to the patient based on patient data. For example, the injection protocol preview depicts a total medical fluid volume of 10.0 ml, a total saline fluid volume of 10.0 ml, and a total injection procedure duration of 10 seconds. The pressure limit for the injection is set (e.g., configured and/or stored in memory) at 325 psi (above which threshold, the injection procedure may be configured to abort). The injection protocol preview further includes a depiction of the phases of the injection procedure, based on the current parameter settings. The phases are shown in a series of rows, with one row for each phase. Each phase includes a fluid identifier (e.g., an icon depicting the type of fluid or lack of fluid), a flow rate, a volume, and a duration of injection. As shown in the depicted example, the current injection protocol prescribes an injection phase with a flow rate of 2 ml/sec, a contrast volume of 10.0 ml, and a contrast injection duration of 5 seconds, followed by a flush phase with a flow rate of 2 ml/sec, a saline volume of 10.0 ml, and a saline injection duration of 5 seconds.

Referring now to FIG. 7B, FIG. 7B illustrates a non-limiting embodiment of a sixth GUI 400f for use in the described methods for operating a fluid injection system. It will be appreciated that the precise style, arrangement, and configuration of interface components of the sixth GUI 400f are for exemplary purposes only and are not to be taken as limiting on the present disclosure. One or more elements of another GUI described herein may be combined with that of the sixth GUI 400f, and one or more elements of the sixth GUI 400f may be included in other GUIs, to provide the features of the described methods for operating a fluid injection system.

The sixth GUI 400f depicts substantially the same illustrative window as the fifth GUI 400e in FIG. 7A, but with the dosing calculator feature enabled. The user may select a button (e.g., a toggle switch) to enable the dosing calculator. Once the dosing calculator is enabled, certain parameters of the injection protocol may be updated and determined based on patient data. For example, the workstation device 106 may identify the concentration of the selected medical fluid (e.g., a contrast fluid with a concentration of 1 mmol/ml) from a programmed data table (e.g., a hard-coded chart), a default dosage (e.g., 0.1 ml/kg, also from a programmed data table), a patient dose (in units ml/kg) based on patient data (e.g., from a programmed data table based on age and/or weight of the patient), and a dosage total volume (in units ml) calculated based on patient data (e.g., determining a patient weight, by converting 187 pounds to 85 kg, and multiplying patient weight by dosage or patient dose to determine a total volume of 8.5 ml). Because the dosing calculator feature was enabled, the injection protocol preview has been updated with a more precise and now customized set of parameters for the injection protocol of the fluid injection procedure. For example, the injection protocol preview depicts a total medical fluid volume of 8.5 ml, a total saline fluid volume of 10.0 ml, and a total injection procedure duration of 9 seconds. The phases have also been updated, to an injection phase with a flow rate of 2 ml/sec, a contrast volume of 8.5 ml, and a contrast injection duration of 4 seconds, followed by a flush phase with a flow rate of 2 ml/sec, a saline volume of 10.0 ml, and a saline injection duration of 5 seconds. In this manner, the parameters of the injection protocol have been automatically generated without user consultation of external tools/manuals. From this point, the user may review and confirm the final parameters of the injection protocol, which may be used to execute the fluid injection procedure by instructing the fluid injection system.

Referring now to FIG. 7C, FIG. 7C illustrates a non-limiting embodiment of a seventh GUI 400g for use in the described methods for operating a fluid injection system. It will be appreciated that the precise style, arrangement, and configuration of interface components of the seventh GUI 400g are for exemplary purposes only and are not to be taken as limiting on the present disclosure. One or more elements of another GUI described herein may be combined with that of the seventh GUI 400g, and one or more elements of the seventh GUI 400g may be included in other GUIs, to provide the features of the described methods for operating a fluid injection system.

The seventh GUI 400g depicts substantially the same illustrative window as the sixth GUI 400f in FIG. 7B, but with the dosing calculator feature fully integrated as part of the preset injection protocol. The seventh GUI 400g, in particular, may be displayed in response to a user selecting the preset “Breast” injection protocol that was shown in the third GUI 400c of FIG. 6A. Patient data may be automatically retrieved from a patient record of the hospital information system 108 and displayed in the seventh GUI 400g. Furthermore, parameters of the injection protocol may be automatically calculated based on the patient data (e.g., patient weight) and displayed as such in the seventh GUI 400g. To that end, a recommended medical fluid is displayed, along with a recommended saline fluid. An injection phase and a flush phase are configured based on the selected anatomical region identifier of “Breast”. Furthermore, the workstation device 106 may identify a dosage total volume (ml) calculated based on patient data (e.g., 8.5 ml, derived from dosage and patient weight). The injection protocol preview, as shown, depicts the automatically generated total medical fluid volume of 8.5 ml, a total saline fluid volume of 20.0 ml, and a total injection procedure duration of 14 seconds. The phases of the planned fluid injection procedure are also broken out, including an injection phase with a flow rate of 2 ml/sec, a contrast volume of 8.5 ml, and a contrast injection duration of 4 seconds, followed by a flush phase with a flow rate of 2 ml/sec, a saline volume of 20.0 ml, and a saline injection duration of 10 seconds. In this manner, the parameters of the injection protocol have been automatically generated without user consultation of external tools/manuals. From this point, the user may review and confirm the final parameters of the injection protocol, which may be used to execute the fluid injection procedure by instructing the fluid injection system.

Referring now to FIG. 7D, FIG. 7D illustrates a non-limiting embodiment of an eighth GUI 400h for use in the described methods for operating a fluid injection system. It will be appreciated that the precise style, arrangement, and configuration of interface components of the eighth GUI 400h are for exemplary purposes only and are not to be taken as limiting on the present disclosure. One or more elements of another GUI described herein may be combined with that of the eighth GUI 400h, and one or more elements of the eighth GUI 400h may be included in other GUIs, to provide the features of the described methods for operating a fluid injection system.

The eighth GUI 400h depicts substantially the same illustrative window as the seventh GUI 400g in FIG. 7C, but for a different patient undergoing a fluid injection procedure based on a cardiac anatomical region. The eighth GUI 400h, in particular, may be displayed in response to a user selecting patient “Wei Zhang” in the second GUI 400b of FIG. 5, followed by the preset “Cardiac” injection protocol shown in the third GUI 400c of FIG. 6A. Patient data may be automatically retrieved from a patient record of the hospital information system 108 and displayed in the eighth GUI 400h. Furthermore, parameters of the injection protocol may be automatically calculated based on the patient data (e.g., patient weight) and displayed as such in the eighth GUI 400h. To that end, a recommended medical fluid is displayed, along with a recommended saline fluid. Because the preset injection protocol for a “Cardiac” anatomical region requires two injection phases and two saline phases separated by a hold phase, the workstation device 106 has automatically generated the phases of a corresponding fluid injection procedure. The injection protocol preview, as shown, depicts the automatically generated total medical fluid volume of 8.4 ml, a total saline fluid volume of 40.0 ml, and a total injection procedure duration of 10 minutes and 12 seconds. The phases of the planned fluid injection procedure are also broken out, including a first injection phase with a flow rate of 4 ml/sec, a contrast volume of 4.2 ml, and a contrast injection duration of 1 second, followed by a first flush phase with a flow rate of 4 ml/sec, a saline volume of 20.0 ml, and a saline injection duration of 5 seconds, followed by a hold phase (e.g., where no fluid is injected) having a duration of 10 minutes, followed by a second injection phase having the same parameters as the first injection phase, and followed by a second flush phase having the same parameters as the first flush phase. In this manner, the parameters of the injection protocol have been automatically generated without user consultation of external tools/manuals. From this point, the user may review and confirm the final parameters of the injection protocol, which may be used to execute the fluid injection procedure by instructing the fluid injection system.

Referring now to FIG. 7E, FIG. 7E illustrates a non-limiting embodiment of a ninth GUI 400i for use in the described methods for operating a fluid injection system. It will be appreciated that the precise style, arrangement, and configuration of interface components of the ninth GUI 400i are for exemplary purposes only and are not to be taken as limiting on the present disclosure. One or more elements of another GUI described herein may be combined with that of the ninth GUI 400i, and one or more elements of the ninth GUI 400i may be included in other GUIs, to provide the features of the described methods for operating a fluid injection system.

The ninth GUI 400i depicts substantially the same illustrative window as the seventh GUI 400g in FIG. 7C, but for a non-identified (e.g., anonymous, unknown) patient. The ninth GUI 400i, in particular, may be displayed in response to a user selecting “Unknown Patient” button shown in the second GUI 400b of FIG. 5, followed by the “Breast” preset injection protocol that was shown in the third GUI 400c of FIG. 6A. Since the “Unknown Patient” button is associated with an identifier that is unassociated with a patient record, patient data is not automatically retrieved from a patient record of the hospital information system 108. Instead, the ninth GUI 400i notifies the user that patient data input is required, including, in particular, patient weight. Once the patient weight data is input by the user, the workstation device 106 may automatically generate parameters for the injection protocol based on the input patient data. The automatically generated parameters may be populated in the preview window for the injection protocol, much the same as in the seventh GUI 400g. From that point, the user may review and confirm the final parameters of the injection protocol, which may be used to execute the fluid injection procedure by instructing the fluid injection system.

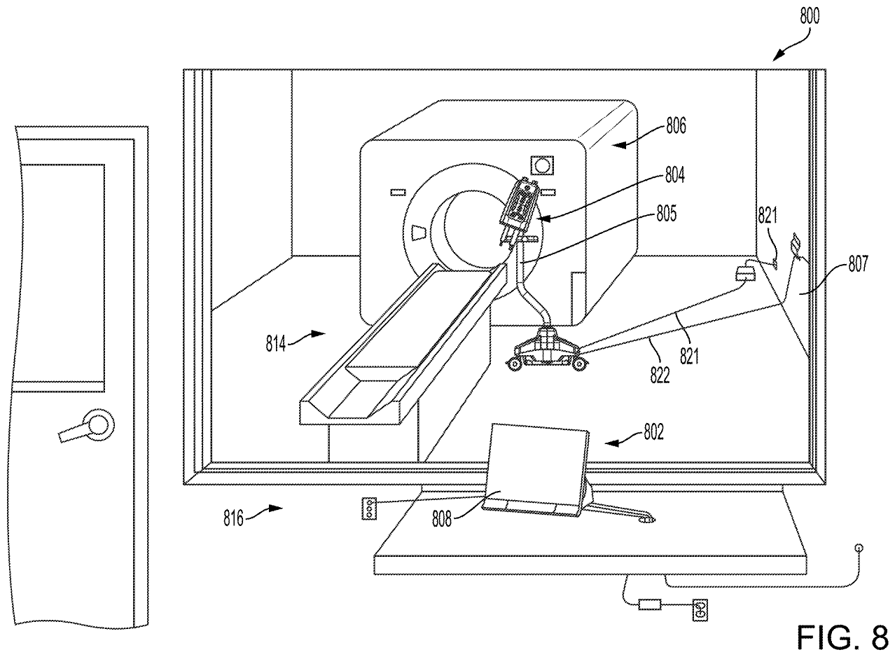

Referring now to FIG. 8, FIG. 8 illustrates a non-limiting embodiment of medical imaging suite 800. Medical imaging suite 800 may be located in hospitals and/or imaging centers for use in performing imaging procedures on patients for the purpose of diagnosing disease and/or other abnormalities. Medical imaging suite 800 may include scan room 814 in which fluid injection system 804 and medical imaging system 806 are located and control room 816 from which an individual (e.g., a Radiologist, a Technologist, or other medical personnel) may monitor a patient through one or more workstation devices 802 associated with fluid injection system 804 and/or medical imaging system 806. Fluid injection system 804 may include a multi-fluid delivery system, such as the MEDRAD® MRXperion MR Injection System offered by Bayer HealthCare LLC. In some non-limiting embodiments, fluid injection system 804 may be the same as or similar to fluid injection system 104. In some non-limiting embodiments, workstation device 802, which includes display unit 808, may be the same as or similar to workstation device 106. In some non-limiting embodiments, injection protocol management system 102 may be a component of workstation device 802. As shown in FIG. 8, workstation device 802 may be used (e.g., by medical personnel) to set up and/or execute a fluid injection procedure for a patient. In some non-limiting embodiments, workstation device 802 may provide a user interface (e.g., an application-based user interface, a web-based user interface, etc.) on display unit 808 for control of fluid injection system 804 and medical imaging system 806. The parameters of an injection protocol of a fluid injection procedure may be automatically generated by injection protocol management system 102, and, in some non-limiting embodiments, displayed on workstation device 802 and/or communicated to fluid injection system 804 for execution of the fluid injection procedure.

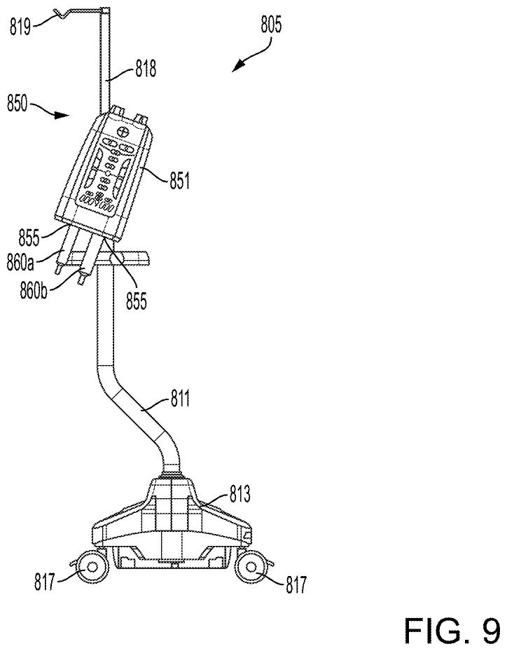

Referring now to FIG. 9, FIG. 9 illustrates an enlarged and partial view of the fluid injection system 804 shown in FIG. 8, specifically a non-limiting embodiment of a scan room unit 805 (e.g., of the MEDRAD® MRXperion MR Injection System). In some non-limiting embodiments, scan room unit 805 includes pedestal 811 mounted to base 813 to which lockable casters 817 are affixed at bottom for use in moving the scan room unit 805 within scan room 814 as needed during imaging procedures. Although primarily intended to support injector head 850 of scan room unit 805, pedestal 811 also includes integrated IV pole 818 inclusive of one or more hooks 819 for hanging IV-related accessories of minimal weight. In some non-limiting embodiments, base 813 houses various electronic and communications components as well as the power supply for scan room unit 805. Among other connections, scan room unit 805 has, as shown in FIG. 8, power cable 821 for conveying through penetration panel 807 electrical power to the power supply and fiber optic link 822 through which to communicate with workstation device 802 in control room 816 to enable control of the operation of fluid injection system 804.

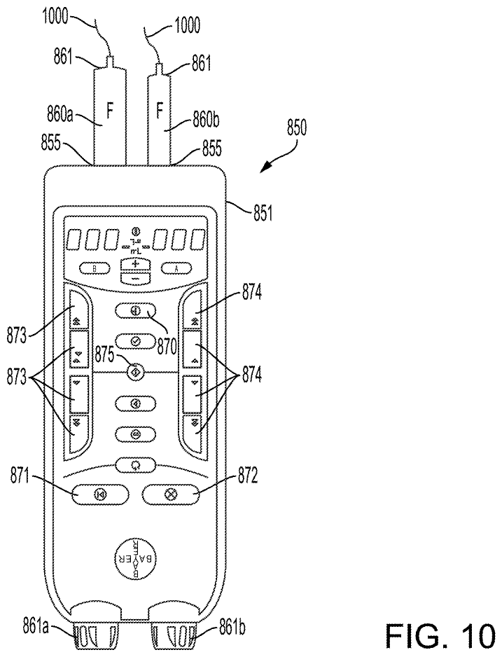

Referring now to FIG. 10, FIG. 10 illustrates a non-limiting embodiment of injector head 850 of scan room unit 805 (e.g., of the MEDRAD® MRXperion MR Injection System) shown in FIG. 9. As shown in FIG. 10, injector head 850 may include housing 851 and at least one fluid reservoir 860a/860b, such as a syringe. In some non-limiting embodiments, a fluid injection system may include a drive component to control fluid flow into or out of a fluid reservoir, such as a piston associated with each of fluid reservoirs 860a/860b that drives its associated plunger within a barrel of fluid reservoir 860a/860b. In some non-limiting embodiments, each of fluid reservoirs 860a/860b is adapted to releasably interface with housing 851 at port 855 therefor. Each fluid reservoir 860a/860b of the fluid injection system is configured to be filled with at least one medical fluid F, such as an imaging contrast media, saline solution, and/or any desired medical fluid. Each fluid reservoir 860a/860b may be filled with a different medical fluid F. In some non-limiting embodiments, fluid injection system 804 may be a multi-syringe injector, as shown, where two fluid reservoirs 860a/860b may be oriented side-by-side or in another spatial relationship and are separately actuated by respective pistons associated with the fluid injection system.

In some non-limiting embodiments, fluid injection system 804 may be used during a medical procedure to inject the at least one medical fluid F into the vasculature of a patient by selectively driving the plunger associated with each fluid reservoir 860a/860b with its respective drive component. For each fluid reservoir, the drive component may move the plunger toward the distal end 861 of the fluid reservoir to expel the fluid F from the fluid reservoir into and through fluid path set 1000 during a priming, purging, and/or fluid delivery step. In some non-limiting embodiments, fluid path set 1000 may include at least one tube or tube set configured to be in fluid communication with each fluid reservoir 860a/860b to place each fluid reservoir in fluid communication with a flexible administration tube and associated catheter for delivering the fluid F from each or both fluid reservoirs 860a/860b to a patient at a vascular access site.

In some non-limiting embodiments, injector head 850 may include a variety of control buttons and indicators through which to monitor and affect operation of fluid injection system 804. For example, manual knobs 861a/861b may be used by an operator to aid in priming of the tube set(s) and to assist in ensuring correct placement of the catheter into the patient as well as to check patency of the fluid connection with the patient. Injector head 850 may also include prime button 870 that, when pressed, activates the priming function and which illuminates to indicate to the operator that priming can begin. Injector head 850 may also include start/hold button 871 that, when pressed, will initiate an injection procedure when fluid injection system 804 has been armed and abort button 872 that, when pressed, will terminate an injection procedure in progress and thereby disarm fluid injection system 804. Injector head 850 may also feature forward and reverse piston control buttons 873 and 874 and an enable piston control button 875 to activate buttons 873 and 874 and thereby enable their use to advance and retract the pistons. In some non-limiting embodiments, injector head 850 may include various other control buttons and indicators, such as those described in MEDRAD® MRXperion MR Injection System Operation Manual, published in January 2021 by Bayer Medical Care Inc., which is incorporated herein by reference.

Although the above systems, methods, and computer program products have been described in detail for the purpose of illustration based on what is currently considered to be the most practical and preferred embodiments, it is to be understood that such detail is solely for that purpose and that the present disclosure is not limited to the described embodiments or aspects but, on the contrary, is intended to cover modifications and equivalent arrangements that are within the spirit and scope of the appended claims. For example, it is to be understood that the present disclosure contemplates that, to the extent possible, at least one feature of any embodiment or aspect can be combined with at least one feature of any other embodiment.

What is claimed is:

1. A system for operating a fluid injection system comprising:
at least one processor programmed or configured to:
receive patient data associated with a patient undergoing a fluid injection procedure, wherein the patient data comprises data associated with a weight of the patient;
automatically generate at least one parameter of an injection protocol for the fluid injection procedure based on the patient data and independent of an input associated with a dosage of a medical fluid for the fluid injection procedure received via a user interface associated with a fluid injection system that is to execute the fluid injection procedure, wherein, when automatically generating the at least one parameter, the at least one processor is programmed or configured to:
determine the dosage of the medical fluid for the fluid injection procedure based on:
the weight of the patient,
a concentration of the medical fluid,
an anatomical region of the patient in which the fluid injection procedure is to take place, or
any combination thereof; and
provide the at least one parameter of the injection protocol to the fluid injection system.

2. The system of claim 1, further comprising a display unit, wherein the at least one processor is further programmed or configured to:
cause a plurality of patient identifiers to be displayed in a user interface on the display unit; and
receive a selection of a patient identifier of the plurality of patient identifiers via the user interface;
wherein, when receiving the patient data, the at least one processor is programmed or configured to:
receive the patient data based on the selection of the patient identifier received via the user interface.

3. The system of claim 1, wherein, when receiving the patient data, the at least one processor is programmed or configured to:
receive the patient data from a user input via a user interface displayed on a display unit.

4. The system of claim 1, wherein the patient data associated with the patient undergoing the fluid injection procedure corresponds to a patient record for the patient stored in a hospital information system, and wherein the at least one processor is further programmed or configured to:
transmit a unique identifier associated with the patient record for the patient to the hospital information system; and
wherein, when receiving the patient data, the at least one processor is programmed or configured to:
receive the patient data from the hospital information system.

5. The system of claim 1, wherein, when automatically generating the at least one parameter of the injection protocol for the fluid injection procedure, the at least one processor is further programmed or configured to:
determine a first dosage of the medical fluid for a first injection phase of the fluid injection procedure;
determine a first flush phase of the fluid injection procedure, wherein the first flush phase comprises a first injection of saline fluid;
determine a second dosage of the medical fluid for a second injection phase of the fluid injection procedure based on at least one of the following:
the weight of the patient,
the concentration of the medical fluid,
the anatomical region, or
any combination thereof; and
determine a second flush phase of the fluid injection procedure, wherein the second flush phase comprises a second injection of saline fluid.

6. The system of claim 5, wherein the at least one processor is further programmed or configured to:
cause the fluid injection procedure to be executed in order of the first injection phase, the first flush phase, a hold phase, the second injection phase, and the second flush phase.

7. The system of claim 5, wherein, when automatically generating the at least one parameter of the injection protocol for the fluid injection procedure, the at least one processor is programmed or configured to:
determine a first flow rate of the medical fluid for the first injection phase based on the concentration of the medical fluid and the anatomical region; and
determine a second flow rate of the medical fluid for the second injection phase based on the concentration of the medical fluid and the anatomical region.

8. The system of claim 1, further comprising a display unit, wherein the at least one processor is further programmed or configured to:
cause a plurality of anatomical region identifiers to be displayed in a user interface on the display unit; and
receive a selection of an anatomical region identifier of the plurality of anatomical region identifiers via the user interface;
wherein, when automatically generating the at least one parameter of the injection protocol for the fluid injection procedure, the at least one processor is programmed or configured to:
determine a type of the medical fluid, a concentration of the medical fluid, and the anatomical region of the patient in which the fluid injection procedure is to take place based on the selection of the anatomical region identifier received via the user interface.

9. A method for operating a fluid injection system, comprising:
receiving, with at least one processor, patient data associated with a patient undergoing a fluid injection procedure, wherein the patient data comprises data associated with a weight of the patient;
automatically generating, with at least one processor, at least one parameter of an injection protocol for the fluid injection procedure and independent of an input associated with a dosage of a medical fluid for the fluid injection procedure received via a user interface associated with a fluid injection system that is to execute the fluid injection procedure based on the patient data, wherein automatically generating the at least one parameter comprises:
determining the dosage of the medical fluid for the fluid injection procedure based on:
the weight of the patient,
a concentration of the medical fluid, or
an anatomical region of the patient in which the fluid injection procedure is to take place; or
any combination thereof; and
providing, with at least one processor, the at least one parameter of the injection protocol.

10. The method of claim 9, further comprising:
causing a plurality of patient identifiers to be displayed in a user interface on a display unit; and
receiving a selection of a patient identifier of the plurality of patient identifiers via the user interface;
wherein receiving the patient data comprises:
receiving the patient data based on the selection of the patient identifier received via the user interface.

11. The method of claim 10, wherein the patient data associated with the patient undergoing the fluid injection procedure corresponds to a patient record for the patient stored in a hospital information system, and wherein the method further comprises:
transmitting a unique identifier associated with the patient record for the patient to the hospital information system; and
wherein receiving the patient data comprises:
receiving the patient data from the hospital information system.

12. The method of claim 9, wherein receiving the patient data comprises:
receiving the patient data from a user input via a user interface displayed on a display unit.

13. The method of claim 9, wherein automatically generating the at least one parameter of the injection protocol for the fluid injection procedure further comprises:
determining a first dosage of the medical fluid for a first injection phase of the fluid injection procedure;
determining a first flush phase of the fluid injection procedure comprising a first injection of a saline fluid;
determining a second dosage of the medical fluid for a second injection phase of the fluid injection procedure based on at least one of the following: the weight of the patient, the concentration of the medical fluid, or the anatomical region; and
determining a second flush phase comprising a second injection of the saline fluid.

14. The method of claim 13, further comprising:
causing execution of the fluid injection procedure in order of the first injection phase, the first flush phase, a hold phase, the second injection phase, and the second flush phase.

15. The method of claim 13, wherein automatically generating the at least one parameter of the injection protocol for the fluid injection procedure further comprises:
determining a first flow rate of the medical fluid for the first injection phase based on the concentration of the medical fluid and the anatomical region of the patient in which the fluid injection procedure is to take place; and
determining a second flow rate of the medical fluid for the second injection phase based on the concentration of the medical fluid and the anatomical region of the patient in which the fluid injection procedure is to take place.

16. The method of claim 9, further comprising:
causing a plurality of anatomical region identifiers to be displayed in a user interface on a display unit; and
receiving a selection of an anatomical region identifier of the plurality of anatomical region identifiers via the user interface;
wherein automatically generating the at least one parameter of the injection protocol for the fluid injection procedure comprises:
determining the medical fluid, the concentration of the medical fluid, and the anatomical region of the patient in which the fluid injection procedure is to take place based on the selection of the anatomical region identifier received via the user interface.

17. A computer program product for operating a fluid injection system, the computer program product comprising at least one non-transitory computer-readable medium including one or more instructions that, when executed by at least one processor, cause the at least one processor to:
receive patient data associated with a patient undergoing a fluid injection procedure, wherein the patient data comprises data associated with a weight of the patient;
automatically generate at least one parameter of an injection protocol for the fluid injection procedure based on the patient data and independent of an input associated with a dosage of a medical fluid for the fluid injection procedure received via a user interface associated with a fluid injection system that is to execute the fluid injection procedure, wherein, the one or more instructions that cause the at least one processor to automatically generate the at least one parameter, cause the at least one processor to:
determine the dosage of the medical fluid for the fluid injection procedure based on at least one of the following:
the weight of the patient,
a concentration of the medical fluid, or
an anatomical region of the patient in which the fluid injection procedure is to take place; and
provide the at least one parameter of the injection protocol to the fluid injection system.

18. The computer program product of claim 17, wherein the one or more instructions further cause the at least one processor to:
cause a plurality of patient identifiers to be displayed in a user interface on a display unit; and
receive a selection of a patient identifier of the plurality of patient identifiers via the user interface;
wherein, the one or more instructions that cause the at least one processor to receive the patient data, cause the at least one processor to:
receive the patient data based on the selection of the patient identifier received via the user interface.

19. The computer program product of claim 17, wherein the patient data associated with the patient undergoing the fluid injection procedure corresponds to a patient record for the patient stored in a hospital information system, and wherein the one or more instructions further cause the at least one processor to:
transmit a unique identifier associated with the patient record for the patient to the hospital information system; and
wherein, the one or more instructions that cause the at least one processor to receive the patient data, cause the at least one processor to:
receive the patient data from the hospital information system.

20. The computer program product of claim 17, wherein, the one or more instructions that cause the at least one processor to automatically generate the at least one parameter of the injection protocol for the fluid injection procedure further cause the at least one processor to:
determine a first dosage of the medical fluid for a first injection phase of the fluid injection procedure;
determine a first flush phase of the fluid injection procedure, wherein the first flush phase comprises a first injection of saline fluid;
determine a second dosage of the medical fluid for a second injection phase of the fluid injection procedure based on at least one of the following:
the weight of the patient,
the concentration of the medical fluid,
the anatomical region, or
any combination thereof; and
determine a second flush phase of the fluid injection procedure, wherein the second flush phase comprises a second injection of saline fluid; and
wherein the one or more instructions further cause the at least one processor to:
cause the fluid injection procedure to be executed in order of the first injection phase, the first flush phase, a hold phase, the second injection phase, and the second flush phase.
